(19) United States
(12) Patent Application Publication
(10) Pub. No.: US 2023/0183697 A1
(43) Pub. Date: Jun. 15, 2023

(54) COMPOSITIONS AND METHODS FOR REGULATION OF CELL ACTIVITY VIA MODULATION OF BETA-CYTOKINE ACTIVITY

(71) Applicant: THE CHILDREN'S HOSPITAL OF PHILADELPHIA, Philadelphia, PA (US)

(72) Inventor: Patrick Viatour, Narberth, PA (US)

(73) Assignee: THE CHILDREN'S HOSPITAL OF PHILADELPHIA, Philadelphia, PA (US)

(21) Appl. No.: 17/996,806

(22) PCT Filed: Apr. 26, 2021
(86) PCT No.: PCT/US2021/029209

§ 371 Date: Oct. 21, 2022

Related U.S. Application Data
(60) Provisional application No. 63/015,442, filed on Apr. 24, 2020.

(51) Int. Cl. C12N 15/113; C07K 16/28; A61P 7/00

(52) U.S. Cl. CPC C12N 15/113; A61P 7/00; C07K 16/2803; C12N 2310/11

(57) ABSTRACT

Compositions and methods for the regulation of the cell cycle are encompassed, wherein the cell cycle is regulated via modulation of βcytokine activity.

### CROSS REFERENCE TO RELATED APPLICATION

This application claims priority to U.S. Provisional Application No. 63/015,442 filed Apr. 24, 2020, which is incorporated herein by reference as though set forth in full.

### FIELD OF THE INVENTION

This invention relates to fields of hematopoietic stress response modulation, and the relationship between cell cycle regulation and cytokine activity effected by E2f factors. More specifically, the invention provides therapeutics for directing cell fate and activity in multipotent hematopoietic stem and progenitor cells (HSPCs) and their myeloid progeny by modulating the expression of desired gene programs.

### BACKGROUND OF THE INVENTION

Several publications and patent documents are cited throughout the specification in order to describe the state of the art to which this invention pertains. Each of these citations is incorporated herein by reference as though set forth in full.

Hematopoiesis is a multi-step process that governs the differentiation of hematopoietic stem cells (HSCs) into an array of effector cells through a series of intermediate progenitors with increasingly restricted cellular plasticity¹. In homeostatic conditions, multipotent long-term (LT)-HSCs and short term (ST)-HSC/MPP1 cells occasionally enter cell cycle to either self-renew or differentiate into lineage-primed multipotent progenitors cells (MPP; megakaryocyte-primed MPP2, granulocyte/ monocyte-primed MPP3 and lymphoid-primed MPP4 ²⁻⁵) that differentiate into lineage-restricted progenitors to ultimately generate fully differentiated myeloid and lymphoid cells with no or limited proliferative capacity.

Several transcription factors coordinate hematopoietic stem and progenitor cell (HSPCs, aka KLS cells) fate to ensure an appropriate balance among effector cells from the different lineages. While Ikaros and Pax5 are key promoters of lymphopoiesis, Pu.1, Cebpa/b/e, Irf4/8, Gata1-3, Lef1, Gf1 and Mitf promote the priming of HSPCs as well as their differentiation into various myeloid progenitors (MPs) and ultimately mature myeloid subpopulations, partitioned into granulocytes (neutrophils, eosinophils, basophils) and monocytes/macrophages^{6, 7-9}. Many of these factors interact with each other to form a tightly connected molecular network. Disrupting this network alters the hematopoietic balance and ultimately skews HSPC differentiation towards specific effector population(s) to the detriment of other mature population(s)¹⁰⁻¹².

An “Inflammatory disease” or “ID” refers to a large number of pathologies characterized by an increased systemic expression of pro-inflammatory cytokines. These cytokines stimulate the recruitment and activity of innate and adaptive immune cells to nurture local and systemic inflammatory conditions. These pathologies are extremely diverse and include an array of cardiovascular and autoimmune diseases, bacterial and viral systemic infections, allergies, cancer, aging, etc, with development ranging from days/weeks to years. The wide time span of these diseases roughly partitions them into acute and chronic inflammatory conditions. It is estimated that ~50% of the American population live with a chronic inflammatory condition (per CDC). Although many of them are not lethal stricto sensu, they are debilitating and represent a major public health concern. In addition, certain severe inflammatory episodes can give rise to “cytokine storms, potentially evolving into multi-organ failure having a substantial fatality rate. For example, cytokine storm is frequently observed in some cases of Covid-19 infection.

Most innate immune cells are short-lived, which necessitates their constant replacement driven by HSPCs. As such, pro-inflammatory cytokines also stimulate HSPC proliferation to drive stress myelopoiesis. Therefore, the short lifespan of pro-inflammatory innate immune cells opens a window of therapeutic opportunity to decrease their production in the context of ID treatments. However, the mechanisms regulating stress myelopoiesis are only partially understood. This important gap in our knowledge represents a major hurdle towards the development of novel strategies to tamper the production of pro-inflammatory innate immune cells for ID therapy.

Clearly, a need exists for new therapeutic approaches for modulating skewed HSPC differentiation to enhance and maintain hematopoietic balance during pathological conditions.

### SUMMARY OF THE INVENTION

In accordance with the present invention, a method for modulating stress hematopoiesis in a subject in need thereof is provided. In an exemplary method an agent which represses myeloid cell formation from differentiating multipotent progenitor cells (MPP) is disclosed, the method alleviating symptoms of stress hematopoiesis in a subject in need. Under homeostasis conditions, MPP cells are induced to differentiate into myeloid cells or lymphoid cells. Hematopoietic stress associated with the pathological conditions described below triggers skewed production of undesirable myeloid cells from differentiating MPP cells. Modulation of MPP cell differentiation is achieved by contacting MPP cells with at least one inhibitor of βcytokine activity thereby repressing undesirable myeloid cell formation and activity and providing therapeutic benefit to the subject. In another aspect, cell proliferation and βcytokine activity enhancement resulting from the activation of the expression of Csf2rb byE2f factors is inhibited by a compound of interest, for example antibodies which block the beta chain receptor or inhibitory nucleic acid molecules which down modulate expression of cytokine-specific α chains of the β cytokine receptor.

In certain embodiments, the MPP cells are one or both of MPP3 and MPP4 cells. In other embodiments, the agent blocks or inhibits the activity of the common β chain of the β cytokine receptor. In other embodiments, the agent blocks or inhibits the activity of the cytokine specific α chain of the β cytokine receptor. The agent can also be an inhibitory nucleic acid which targets the Csf2rb gene. Alternatively, the inhibitory nucleic acid targets the cytokine-specific α chain gene of the β cytokine receptor. Preferred inhibitory nucleic acids include, without limitation, an siRNA, an shRNA, and an antisense oligonucleotide.

In another embodiment, βcytokine activity is inhibited via administration of at least one antibody which blocks IL3 binding to the cytokine specific α chain, an antibody that blocks Gm-CsF binding to the α chain or both. Small molecule inhibitors of the βcytokine receptor may also be administered. These can include administration of at least one of Csl311, Csl362, and Mavrilimumab. The method can also further comprise administration of at least one NSAIDS.

In particularly preferred embodiments, the method described above is effective to modulate hematopoiesis in subject in need thereof who are at risk for, or have disorders associated with hematopoietic stress in a therapeutically beneficial fashion. Such disorders include, without limitation, systemic infection, chronic inflammation, e.g., lupus, Crohn’s disease and arthritis, aging, hypercholesterolemia, myocardial infarction, lupus, ischemia, and arthritis.

### BRIEF DESCRIPTION OF THE DRAWINGS

FIGS. 1A -1C. Rb family inactivation drives monocytosis. FIGS. 1A-1B Flow cytometry analysis of myeloid populations (characterized by expression of the Mac1⁺ marker) in CT and TKO mice. Mac1+ cells are further fractioned into inflammatory subpopulations on the basis of Ly6c (monocyte marker), Ly6g (granulocyte marker) and Rank (osteoclast marker). FIG. 1A representative Flow cytometry panels; FIG. 1B Quantification (n=3). FIG. 1C H&E staining of sections from femur from CT and TKO mice. Arrowheads indicate osteoclastic activity.

FIGS. 2A -2H. Proliferation alters the self-renewal potential of “long term″-hematopoietic stem cells (LT-HSCs) without affecting their cell fate decision. FIG. 2A UMAP display of single-cell RNA sequencing (sc-Seq) analysis for 3,081 CT (upper panel) and 3,208 TKO (lower panel) LT-HSCs identifies 5 cellular clusters in each population. FIG. 2B heatmap for the expression of representative genes for different steps of hematopoiesis shows increased MPP gene expression in the most primitive TKO LT-HSC cluster and overall increased proliferation gene expression in TKO LT-HSC versus their CT counterparts. FIGS. 2C-2D Trajectory analysis by RNA Velocity and Monocles-3D. CT-LT-HSC are spread through a linear trajectory while TKO LT-HSC show several trajectories merging into cluster 1. FIG. 2E SCENIC analysis of CT and TKO LT-HSC identifies transcriptional networks uniquely active in cells found in the most primitive cluster from CT LT-HSC, as well as the pattern of their activity in TKO LT-HSC. Transcriptional networks with unaltered activity, altered activity and absent activity pattern are framed in green, blue and red, respectively. FIG. 2F sc-Seq analysis for CT and TKO LT-HSC were merged for analysis and individually displayed to determine the distribution of the respective populations within the merged population. FIGS. 2G-2H Trajectory analysis by RNA Velocity (FIG. 2G) and Monocles-3D (FIG. 2H) for the merged CT/TKO LT-HSC scRNA-Seq dataset.

FIGS. 3A - 3K. Proliferation alters the function and identity of MPP4 cells. FIG. 3A-FIG. 3B Using surface markers that enable the identification of LT-HSC and MPP1-4 populations (KLS, Cd48, Cd150, Flk2-see⁶⁵ for the comprehensive surface marker combination), our flow cytometry analysis (representative panel in FIG. 3A) shows the frequencies for CT and TKO MPP1-MPP4(FIG. 3B) (CT, white bars; TKO, black bars. n=7). FIG. 3C 100 cells from each subpopulation were plated into standard methylcellulose. The number and identity of colonies were quantified after 8 days in culture (n=3). FIG. 3D CT and TKO MPP1-MPP4 cells were co-cultured with OP9 cells and the number of B cells (Cd45⁺, B220⁺) and Myeloid cells (Cd45⁺, Mac1⁺) were quantified by flow cytometry after 10 days in culture (n=4). FIG. 3E Upon transplantation and Tamoxifen treatment, CT and TKO MPP4 frequency in the BM of recipient mice is evaluated at different time points (n=4). FIG. 3F 100 CT and TKO MPP4 isolated from chimeric mice at 2, and 16 weeks, post Tamoxifen treatment were plated into methylcellulose. Colonies were assessed after 8 days in culture (n=3). FIG. 3G UMAP display of sc-Seq analysis for CT (upper panel, 635 cells) and TKO (lower panel, 401 cells) MPP4 identifies 4 clusters in each population. FIG. 3H heatmap for the expression of representative genes for different steps of hematopoiesis shows that altered gene expression is largely restricted to cluster 2 in TKO MPP4, compared to CT. FIG. 3I Trajectory analysis by Monocles-2D identifies a bipotential (myeloid/lymphoid) fate in CT MPP4 (upper panel) while the lymphoid fate is obliterated in TKO MPP4 (lower panel). FIG. 3J Integration of sc-Seq analysis for individual CT MPP1 (ST-HSC), MPP2 (Meg/E primed), MPP3 (M-primed), MPP4 (L-primed) and TKO-MPP4 identifies 9 different clusters with distinct differentiation profiles. FIG. 3K heatmap for the expression of representative genes for different steps of hematopoiesis.

FIGS. 4A -4G. Accelerated differentiation of TKO MPP4 through myelopoiesis. FIG. 4A Immunophenotypic analysis of MP subpopulations in CT and TKO mice two weeks after Tamoxifen treatment shows increased frequency of CMP-Flt3⁻ cells and decreased frequency of Monocyte Dendritic Progenitors (MDP) (n=3). Bold brown arrow indicates a differentiation pathway that is dominant in TKO mice compared to the alternative pathway that is marked by a blue dotted line. FIG. 4B Colony assay for 100 CT/TKO CMP-Flt3⁺ Cd115^{lo} or GMP cells. Colony identity was determined after 9 days in culture (n=3). FIG. 4C Histological analysis of colony cells described in FIG. 4B identified an increased frequency of monocytes in TKO CMP-Flt3⁺ Cd115^{lo}, but not in TKO GMP, compared to their CT counterparts. FIG. 4D-FIG. 4E Seurat analysis (UMAP in FIG. 4E) of CT (1,979 cells) and TKO (1,149 cells) MPs identifies an alteration of their distribution. CMP-like, GMP-like and MEP-like are identified based on gene expression in the heatmap (FIG. 4D). FIG. 4F Trajectory analysis by Monocles-3D. In CT MP (upper panel), the differentiation towards MEP is short and linear while the differentiation towards GMP is longer and circumvoluted, suggesting a high plasticity among CT GMP subpopulations. In TKO MP (lower panel), the differentiation towards MEP is much longer while the differentiation towards GMP is brief and linear. FIG. 4G Integrated UMAP display of sc-Seq analysis for CT or TKO LT-HSC/MPP4 and MP (upper panel: CT; lower panel: TKO).

FIGS. 5A - 5K. Activation of Csf2rb expression by E2f enhances Pcytokine signaling in TKO HSPCs. FIG. 5A 100 CT and TKO MPP4 cells were plated in cytokine-free media, supplemented with either a cocktail of Il3, Il6 and Scf, or Il3, Scf and Il6 only (n=3). FIG. 5B Representative pictures of colonies from FIG. 5A. FIG. 5C CT and TKO HSPCs were isolated two weeks after Tamoxifen treatment, serum starved and stimulated with Il3 or Gm-Csf (10 ng/ml) for 10 min. p-Erk was used as a readout for βcytokine signaling activity. FIG. 5D Venn diagram display of microarray expression analysis in CT and TKO progenitors: pink circle: LT-HSC ; blue circle: HSPC cells. The figures indicate the respective number of genes upregulated in TKO vs CT cells. Csf2rb increased expression is observed in all TKO HSPC subpopulations while Socs3 increased expression is restricted to TKO LT-HSC. FIG. 5E Upper panel: analysis of mouse and human Csf2rb regulatory region identifies a conserved E2f binding site in the first untranslated exon. Black bars indicate primers for ChIP analysis. Lower panel: ChIP analysis performed on unfractioned BM cells show that E2f1 and E2f3 bind to a region of the Csf2rb promoter that harbors the putative E2f binding site in primary BM cells (n=3). FIGS. 5F-5G Representative expression of the β chain receptor (the protein product of Csf2rb) at the surface of CT and TKO KLS/HSC/MPP1-4 subpopulations (FIG. 5F) as well as myeloid progenitor (MP), granulocytes and monocytes (FIG. 5G), as detected by flow analysis(n=3). FIG. 5H 100CT and TKO LT-HSC (left) and MPP3(right) cells were plated in cytokine-free media, supplemented with Il3 (n=3). Colonies were counted after 8 days in culture. FIG. 5I UMAP visualization of Csf2rb expression in individual CT MP/MPP4 (left) and TKO MP/MPP4 (right) cells. Cells within the black circle represent the most primitive populations. A higher magnification and a violin plot-based quantification of Csf2rb expression for cells within the black circle is displayed. FIG. 5J. MPP4 cells were infected with p-Sicor-RFP lentiviral vectors expressing either a scramble hairpin or two hairpins (a and b) targeted against Csf2rb. RFP⁺ cells were plated for methylcellulose culture and colonies were quantified after 9 days in culture (n=3). FIG. 5K 100 CT/TKO MPP4 cells were plated in methylcellulose with increasing concentration of CSL311®, a specific inhibitor for the common βsubunit receptor. TKO MPP4 growth was inhibited a higher inhibitor concentration, indicative of a stronger pathway activity.

FIGS. 6A - 6J. Pcytokine signaling activates Irf8 expression in TKO MPP4 to drive stress myelopoiesis. FIG. 6A RT-qPCR screening for the expression of transcription factors in CT and TKO MPP4 reveals the increased abundance of Irf8 transcripts in TKO MPP4, compared to CT MPP4 (n=7). FIG. 6B t-SNE display of SCENIC analysis in a binary mode (active vs inactive) shows Irf8 transcriptional network activity in CT MPP4 (top left), TKO MPP4 (top right), CT MP (bottom left) and TKO MP (bottom right). FIG. 6C heatmap for the ATAC-Seq analysis of CT and TKO MPP4 cells isolated two weeks after Rb family loss. The upper panels indicate the number (y axis) of opened region based on their length (x axis) for each population. FIG. 6D pie chart display of opened chromatin regions (peaks) that are unique to either CT or TKO MPP4, based on their genomic location. FIG. 6E UMAP visualization of Irf8 expression in individual CT MP/MPP4 (left) and TKO MP/MPP4 (right) cells. Cells within the black circle represent the most primitive populations. A higher magnification and a violin plot-based quantification of Irf8 expression for cells within the black circle is displayed. FIG. 6F Violin plot for the expression of Irf8 in the four clusters composing CT MPP4 (upper panel) and TKO MPP4 (lower panel). FIG. 6G CT MPP4 were serum starved for two hours, followed by stimulation with 10 ng/ml of Gm-Csf for 15 or 60 minutes. Irf8 expression was detected by RT-qPCR (n=2). FIG. 6H CT and TKO MPP4 cells were infected with p-Sicor-RFP lentiviral vectors expressing either a scramble hairpin or two hairpins (a and b) targeting Irf8. RFP⁺ cells were plated in methylcellulose and colonies were quantified after 9 days in culture (n=3). FIGS. 6I-6J cells from colonies formed during the assay displayed in FIG. 5J (Csf2rb silencing) and FIG. 6H (Irf8 silencing) were counted (FIG. 6I) and cytospun for classification by histological analysis (FIG. 6J). Results from hairpins targeting the same gene (a/b) were averaged in FIG. 6J for easier visualization (n=3).

FIGS. 7A - 7P. Genetic disruption of the E2f/Csf2rb axis impairs stress myelopoiesis. FIG. 7A Left: 100 wild type and Csf2rb deficient (^{-/-}) MPP3 and MPP4 cells were plated in methylcellulose to generate a proliferative stress. The number of colonies was quantified after 9 days in culture (n=6); Right: 100 wild type and Csf2rb deficient (^{-/-}) LT-HSC, MPP1 and MPP2 cells were plated in methylcellulose to generate a proliferative stress. The number of colonies was quantified after 9 days in culture (n=3). FIG. 7B wild-type and Csf2rb deficient mice were injected with either vehicle or recombinant Il1β (0.5ugr/day) every day for 10 days to generate a cytokine stress. FIGS. 7C-7F frequency of granulocytes (FIG. 7C), monocytes (FIG. 7D), Mac1⁺ cells (FIG. 7E) and lymphocytes (FIG. 7F) in the BM of wild-type (White Bar) and Csf2rb deficient (Black Bar) mice exposed to Il1β stress. FIGS. 7G-7I quantification of MPP4 (FIG. 7G), MPP3 (FIG. 7H) and LT-HSC (FIG. 7I) frequency in the BM of wild-type and Csf2rb deficient mice exposed to Il1β stress. FIG. 7J wild-type and Csf2rb deficient mice were injected with either vehicle or recombinant Il1β (0.5ugr/day) once a day for 50 days to generate a chronic cytokine stress. FIGS. 7K-7N frequency of granulocytes (FIG. 7K), monocytes (FIG. 7L), Mac1+ cells (FIG. 7M) and lymphocytes (FIG. 7N) in the BM of wild-type (White Bar) and Csf2rb deficient (Black Bar) mice exposed to chronic Il1β stress. FIGS. 7O-7R quantification of KLS (FIG. 7O), MPP4 (FIG. 7P), MPP3 (FIG. 7Q) and LT-HSC (FIG. 7R) cell frequency in the BM of wild-type and Csf2rb deficient mice exposed to chronic Il1β stress.

FIGS. 8A - 8X. Genetic and pharmacological inhibition of β cytokine signaling decreases inflammation in a mouse model of colitis. FIG. 8A DSS (Dextran Sulfate Sodium) 2% was added to the drinking water of wild-type and Csf2rb deficient mice for 12 days. For FIGS. 8B-8G, the white bar represents wild type mice and the black bar represents Csf2rb deficient mice. FIG. 8B Mice were weighted at the end of the 12-day period (n=5). FIGS. 8C-8E frequency of granulocytes (FIG. 8C), monocytes (FIG. 8D) and lymphocytes (FIG. 8E) in the BM of wild-type and Csf2rb deficient mice. FIGS. 8F-8G number of KLS (FIG. 8F) and MPP4 (FIG. 8G) cells per million of BM cells. FIG. 8H DSS 2% was added to the drinking water of wild-type mice for 12 days. Mice were injected with various blocking antibodies (isotype, anti-GM-Csf, anti-Il3, combination of anti-GM-Csf and anti-Il3) on day 1, 4, 7 and 10 of colitis-inducing DSS treatment. Untreated wild-type mice were used as control. FIG. 8I-8L frequency of granulocytes (FIG. 8I), monocytes (FIG. 8J), Mac1⁺ cells (FIG. 8K) and lymphocytes (FIG. 8L) in the BM. FIG. 8M wild-type and Csf2rb deficient mice were exposed to three cycles of DSS 2% treatment (1 cycle: 12 days with DSS 2% containing drinking water followed by 12 days with normal drinking water). For FIGS. 8N-8V, the white bar represents wild type mice and the black bar represents Csf2rb deficient mice. FIG. 8N Mice were weighted at the end of the treatment (n=5). FIGS. 8O-8R frequency of granulocytes (FIG. 8O), monocytes (FIG. 8P), Mac1+ (FIG. 8Q) and lymphocytes (FIG. 8R) in the BM of wild-type and Csf2rb deficient mice. FIGS. 8S-8V number of KLS (FIG. 8S), MPP4 (FIG. 8T), MPP3 (FIG. 8U) and LT-HSC (FIG. 8V) cells per million of BM cells. FIG. 8W representative histology for intestine classified as “worse” and “fair” by the pathologist. FIG. 8X double-blind repartition of wild-type and Csf2rb^{-/-} mice in the “worse” and “fair” groups.

FIGS. 9A -9K. Genetic and pharmacological inhibition of Pcytokine signaling decreases inflammation in a mouse model of systemic infection. FIG. 9A wild-type mice were injected daily with LPS (1 mgr/kg) for 4 days to recapitulate an acute systemic infection. Mice were injected with different inhibitors on day 1 and 3 of the regimen. FIG. 9B Mice were weighted at the end of the regimen (n=4). FIGS. 9C-9D frequency of granulocytes (FIG. 9C) and monocytes (FIG. 9D) in the BM of mice from the different subgroups (n=4). FIG. 9E wild-type and Csf2rb deficient mice were injected with LPS (1 mgr/kg) every day for 50 days to generate a chronic systemic infection. For FIGS. 9F-9H, the white bar represents wild type mice and the black bar represents Csf2rb deficient mice. FIG. 9F Mice were weighted at the end of the regimen (n=6). FIGS. 9G-9H frequency of granulocytes (FIG. 9G) and monocytes (FIG. 9H) in the BM of mice from the different subgroups (n=6). FIG. 9I Representative H&E staining of intestine sections of wild-type and Csf2rb deficient mice. The ratio of villae/crypt is higher in Csf2rb deficient mice compared to wild-type. FIG. 9J representative pictures of scars (red dotted circles) developing at the site of injection in wild-type and Csf2rb deficient mice. FIG. 9K Representative H&E staining of skin sections collected from the site of injections of wild-type and Csf2rb deficient mice. Scarification is visible in wild-type skin, but not in Csf2rb deficient skin.

FIGS. 10A - 10B. Model. FIG. 10A In homeostatic conditions (upper panel), E2f transcriptional output is mostly limited to the regulation of cell cycle genes. MPP4 cells are lymphoid-primed progenitors that predominantly differentiate into lymphoid cells while myeloid-primed MPP3 cells predominantly differentiate into neutrophils. In inflammatory conditions (lower panel), E2f coordinates the activation of a cell cycle program with the transactivation of Csf2rb, establishing enhanced bcytokine activity as a pre-programmed response of HSPCs to inflammation. Enhanced bcytokine alters the cell fate of MPP4 cells towards monocytosis and increases the myeloid output of MPP3 cells. In addition, stress-specific signaling further modulates the cellular output of proliferative HSPCs. FIG. 10B Strategies for repressing formation and activity of myeloid cells generated from differentiation of said myeloid cells, thereby alleviating the symptoms of stress hematopoiesis.

### DETAILED DESCRIPTION

Hematopoietic stem and progenitor cells (HSPCs) constantly regenerate the hematopoietic system to differentiate into mature effector cells from the myeloid and lymphoid lineages. Multiple hematopoietic stresses of distinct natures stimulate HSPC proliferation and systematically enforce their differentiation towards the myeloid lineage. The shared cellular outcome in response to distinct extracellular cues suggests that the cell cycle status of HSPCs is connected to their cell fate. Indeed, inactivation of negative regulators of cell cycle activity is sufficient to trigger HSPC entry into the cell cycle and enforce their myeloid differentiation, further supporting the concept that a cell intrinsic mechanism links cell cycle activity and cell fate in HSPCs. We find that E2f factors coordinate the transactivation of a cell cycle program and Csf2rb, the common βchain receptor for βcytokines, leading to enhanced βcytokine activity in MPPs and their cellular progeny. The resulting enhanced βcytokine activity triggers Irƒ8 expression in lymphoid-primed MPP4 and enforces their differentiation towards the increased production of myeloid cells from the monocytes lineages. In addition, enhanced βcytokine activity also increases the cellular output of myeloid-primed MPP3. Inactivation of Csf2rb expression impairs the hematopoietic output of MPP4 cells upon exposure to proliferative and cytokine stresses. In addition, genetic and pharmacological inhibition of βcytokine signaling represses the development of inflammatory diseases such as inflammatory bowel disease and systemic infection.

### Definitions

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

A “sample” refers to a sample from a subject that may be tested. The sample may comprise cells, and it may comprise body fluids, such as blood, serum, plasma, cerebral spinal fluid, urine, saliva, tears, pleural fluid, and the like.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The term “Hematopoietic stem cell” or “HSC” refers to a heterogeneous mixture of undifferentiated primitive stem cells, mainly composed of long-term HSCs (“LT-HSCs”) and short-term HSCs (“ST-HSCs”). LT-HSCs are undifferentiated stem cells that have the capacity for self-renewal throughout the life span of an organism. ST-HSCs are undifferentiated stem cells that have the capacity for self-renewal for a limited time prior to full differentiation into a specific lineage. For example, HSCs can differentiate into hematopoietic progenitor cells (“HPCs”) that can further differentiate into clonogenic cells, or cells of a single lineage, that represent a subset of a hematopoietic lineage. HSCs include non-embryonic stem cells isolated from post-natal animals, which are known as adult stem cells. HSCs are isolated from the bone marrows of mammals that are capable of differentiating into ectodermal lineages of blood cells. Furthermore, HSCs are a subset of undifferentiated cells that resides predominantsly in the bone marrow of adult mammals. HSCs, as a population, are capable of self-renewal by maintaining a sufficient number of HSCs within an organism’s bone marrow as a reservoir of uncommitted cells that can be further differentiated into various types of new blood cells. Such newly generated blood cells emerge from the bone marrow and enter the circulatory system in order to continuously replace mature/aging circulating blood cell types. The ability of HSCs, as a population, to differentiate and to give rise to cells of multi-lineages is critical for the preservation of an organism. HSCs that express the combination of human counterparts for these markers are preferably contemplated.

The term “Hematopoiesis” refers to the process of blood cell formation whereby red and white blood cells are replaced through the division of HSCs.

The phrase “Hematopoietic stress” or “stress hematopoiesis” or “stress myelopoiesis” refers to the immune response that is induced when the need for new blood and immune cells exceeds their steady-state production. Reduced levels of Red Blood Cells (RBCs) or platelets, as well as various immune stimuli, can call for urgent hematopoiesis. Hematopoietic stress may activate HSC proliferation after chronic exposure to a cytokine stress. Examples of chronic stressors include, without limitation, infection, inflammation, aging, hypercholesterolemia, myocardial infarction, ischemia, and social factors. Hematopoietic stress invariably triggers the increased proliferation and biased differentiation of HSPCs towards specific mature myeloid subpopulation(s), to the detriment of the lymphoid lineage.

“Hematopoietic cell lineage” generally refers to a particular lineage of differentiated hematopoietic cells, such as myeloid or lymphoid, but could also refer to more differentiated lineages such as dendritic, erythroid, etc.

“Hematopoietic progenitor cells” or “progenitor cells” are generally the first cells to differentiate from (i.e., mature from) blood stem cells; they then differentiate (mature) into the various blood cell types and lineages.

The term “multipotent” refers to the ability of a cell to differentiate into a plurality of different phenotypes. Multipotent cells can generally only differentiate into cells of a single germ layer lineage. This is in contrast to pluripotent cells which can, by definition, differentiate into cells of all three germ layers. Pluripotent cells are characterized primarily by their ability to differentiate to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. A pluripotent cell typically has the potential to divide in vitro for a long period of time, e.g., greater than one year or more than 30 passages.

The term “MPP4” refers to multipotent progenitors immediately downstream of HSCs. While they are primed to differentiate into lymphoid cells, they also retain the capacity to differentiate into myeloid cells in physiological and pathological contexts.

“Lymphoid blood cell” or “Lymphoid cell” are those hematopoietic precursor cells which are able to differentiate to form lymphocytes (B-cells or T-cells). Likewise, “lymphopoeisis” is the formation of lymphocytes.

“Erythroid blood cell” or “Erythroid cell” are those hematopoietic precursor cells which are able to differentiate to form erythrocytes (red blood cells) and “erythropoeisis” is the formation of erythrocytes.

The phrase “myeloid blood cell” or “myeloid cell”, for the purposes herein, encompasses all hematopoietic cells, other than lymphoid and erythroid blood cell lineages as defined above, and “myelopoiesis” involves the formation of blood cells (other than lymphocytes and erythrocytes).

The term “cytokine” is a generic term for proteins released by one cell population which act on another cell as intercellular mediators. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -y; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; and other polypeptide factors including leukemia inhibit factor (LIF) and kit ligand (KL). As used herein, the term cytokine includes proteins from natural sources or from recombinant cell culture and biologically active equivalents of the native sequence cytokines.

The term βcytokine as used herein refers to Il3, Il5, and Gm-Csf.

Csf2rb is a gene that codes for the beta-chain receptor of the βcytokine family (Il3, Il5 and Gm-Csf). βcytokines bind to their own alpha-chain receptor (Il3ra, Il5ra and Csf2ra) and these alpha-chain receptors bind to the common (i.e. shared) beta-chain receptor that is necessary to transmit the intracellular signaling triggered by the binding of the βcytokine to its alpha-chain receptor. An exemplary human Csf2rb variant is Genbank accession number NM_000395.3. Exemplary murine Csf2rb variants are The Genbank accession numbers NM_001358854.1 and NM_007780.4. Exemplary Human Il3ra variants are Genebank accession numbers NM_002183.4 and 001267713.2. An exemplary murine Il3ra variant is Genbank accession number NM_008369.1. An exemplary human Csf2ra variant is Genbank accession number NM_006140.6. An exemplary murine Csf2ra variant is Genbank accession number NM_009970.2.

The term “Interferon regulatory factor 8” or “Irƒ8” refers to a transcription factor that plays a critical role in the regulation of lineage commitment. Irf8 expression is indispensable for myeloid commitment.

The term “E2F factor” or “E2F transcription factor” refers to a family of basic helix-loop-helix transcription factors that control expression of a variety of genes involved in cell cycle regulation. E2F transcription factors typically form heterodimeric complexes with transcription factor DP1 or transcription factor DP2, and they have N-terminal DNA binding and dimerization domains. E2F transcription factors can act as mediators of transcriptional repression or transcriptional activation. E2F factors play a major role during the G₁/S transition in the cell cycle. The E2F factors are generally split by function into two groups. Transcription activators, consisting of E2F1, E2F2, and E2F3a which promote passage through the cell cycle. Transcription repressors, consisting of E2Fb, E2F4, E2F5, E2F6, E2F7, and E2F8 inhibit the cell cycle. Levels of E2F activators fluctuate during the cell cycle, with maximal expression during G₁/S. Levels of E2F repressors remain relatively constant during the cell cycle. The balance between repressor and activator E2Fs help regulate cell cycle progression.

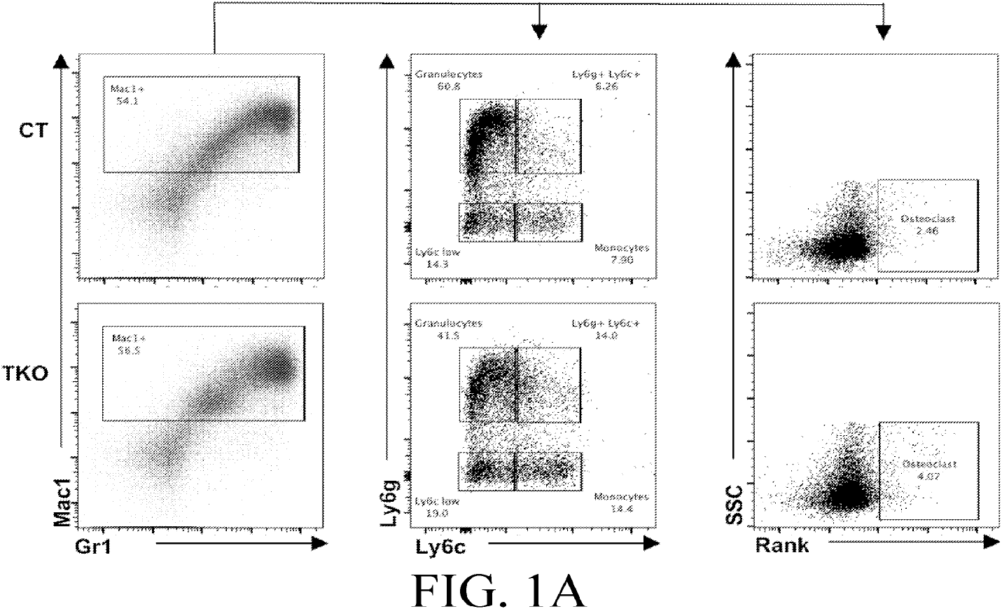

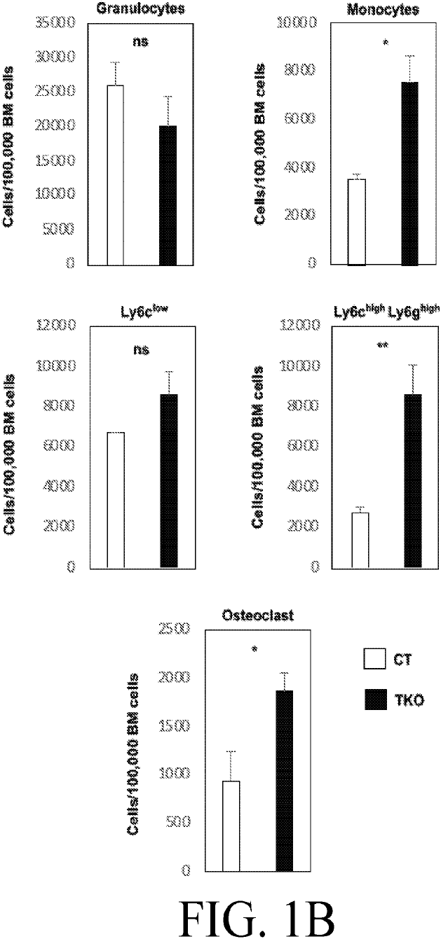

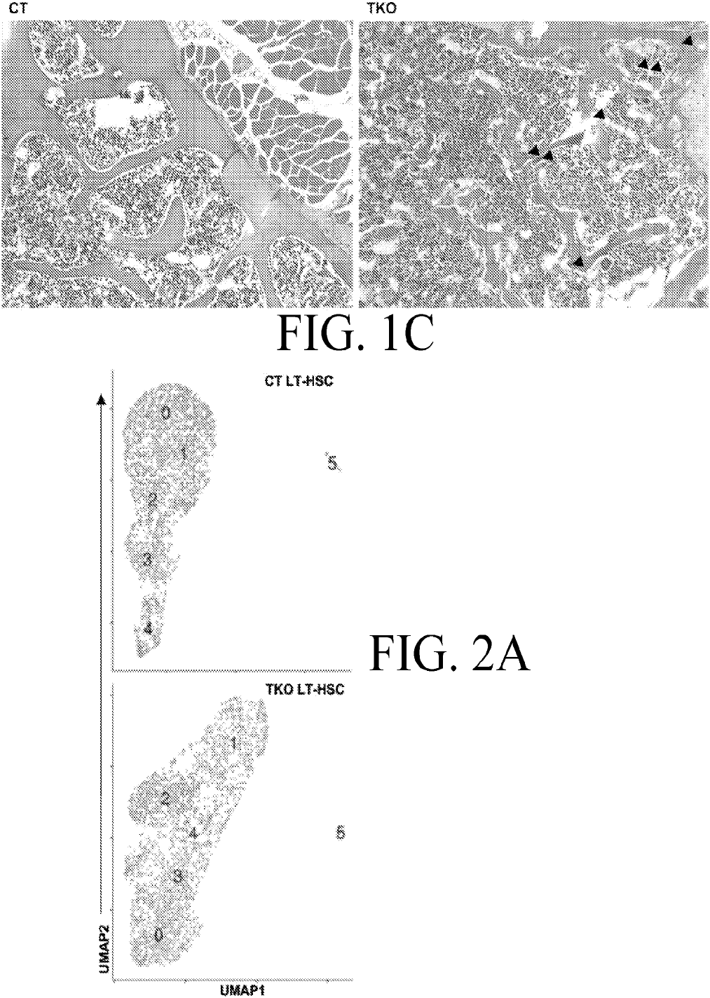

The term “Rb family proteins” refers to a family of proteins that function as regulators of cell cycle progression and suppressors of cellular growth and proliferation. Rb family proteins enforce cell cycle arrest and maintain cellular quiescence by repressing the activity of the E2f family of transcription factors^{20-24,} ²⁵⁻³⁰. Stimulation of quiescent cells by mitogens triggers the transactivation of Cyclin genes and the subsequent stabilization of Cyclin/Cdk complexes. These complexes phosphorylate Rb family proteins, which disrupts their direct interaction with E2f factors and initiates the activation of a cell cycle program by these E2f factors (FIG. 1). The cell autonomous disruption of the Rb/E2f interaction by genetic targeting of negative regulators of proliferation (p21³¹, p27/p57^{32,33}, Pten³⁴, Mek1³⁵, Foxo family³⁶) or overexpression of the Cyclind⅟Cdk4 complex³⁷ triggers a sustained HSC proliferation (eventually followed by their exhaustion), a rapid myeloid expansion (myeloid leukemia, myeloproliferation or a myeloid expansion, based on the nature of the initial genetic hit) and a decreased lymphopoiesis. In addition, in vivo labeling approaches (H2B-GFP³⁸, RA-CFP³⁹) have indicated a link between the proliferation history and the myeloid-biased cell fate of HSPCs. These studies support the concept that a cell intrinsic mechanism (i.e independent of extrinsic stimuli) coordinates the proliferation of HSPCs with their enforced differentiation towards myeloid subpopulations.

The term “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation can also be promoted by release from the actions of intra- or extracellular signals and mechanisms that block or negatively affect cell proliferation.

The terms “renewal” or “self-renewal” or “proliferation” are used interchangeably herein, and refers to a process of a cell making more copies of itself (e.g. duplication) of the cell. In some embodiments, lung progenitor cells are capable of renewal of themselves by dividing into the same undifferentiated cells (e.g. as determined by measuring the presence of absence of one or more cell surface markers) over long periods, and/or many months to years. In some instances, proliferation refers to the expansion of lung progenitor cells by the repeated division of single cells into two identical daughter cells.

The term “positive selection” as used herein refers to selection of a desired cell type by retaining the cells of interest. In some embodiments, positive selection involves the use of an agent to assist in retaining the cells of interest, e.g., use of a positive selection agent such as an antibody which has specific binding affinity for a surface antigen on the desired or target cell. In some embodiments, positive selection can occur in the absence of a positive selection agent, e.g., in a “touch-free” or closed system, for example, where positive selection of a target cell type is based on any of cell size, density and/or morphology of the target cell type.

The term “negative selection” as used herein refers to selection of undesired or non-target stem cells for depletion or discarding, thereby retaining (and thus enriching) the desired target cell type. In some embodiments, negative selection involves the use of an agent to assist in selecting undesirable cells for discarding, e.g., use of a negative selection agent such as a monoclonal antibody which has specific binding affinity for a surface antigen on unwanted or non-target cells. In some embodiments, negative selection does not involve a negative selection agent. In some embodiments, negative selection can occur in the absence of a negative selection agent, e.g., in a “touch-free” or closed system, for example, where negative selection of an undesired (non-target) cell type to be discarded is based on any of cell size, density and/or morphology of the undesired (non-target) cell type.

The phrase “chronic inflammation” refers to a prolonged inflammatory response that involves a progressive change in the type of cells present at the site of inflammation. It is characterized by the simultaneous destruction and repair of the tissue from the inflammatory process. It can follow an acute form of inflammation or be a prolonged low-grade form. The body’s response to chronic inflammation can damage heathy cells, organs, and tissues. Chronic inflammation can be caused by (1) disease, including, without limitation, cancer, heart disease, arthritis, diabetes, obesity, asthma, neurodegenerative diseases, and autoimmune disease, and (2) lifestyle factors including, without limitation, smoking, obesity, alcohol consumption, and chronic stress. In some cases of chronic inflammation, there is not a clear underlying cause. Symptoms including, without limitation, pain, redness, swelling, fatigue, fever, mouth sores, rashes, abdominal pain, and chest pain are commonly caused by chronic inflammation.

The phrase “systemic infection” refers to an infection where the pathogen or symptoms of the infection are spread throughout several systems of the body. Systemic infections are not necessarily more severe than local infections, but affect a larger proportion of the body. The phrase “local infection” refers to an infection where the pathogen or symptoms of the infection is confined to one area of the body. A local infection may progress to a systemic infection. For example, a case of pneumonia might begin in one or both lungs. The microbe responsible for the pneumonia might enter the bloodstream or lymphatic system and be carried to other parts of the body.

The term “monocytosis” refers to an increase in the number of monocytes circulating in the blood. Monocytosis is associated with numerous conditions because of its role in acute and chronic inflammation and infections, immunologic conditions, hypersensitivity reactions, and tissue repair.

The phrase “Inflammatory Bowel Disease” or “IBD” refers to disorders, such as Crohn’s disease and Ulcerative Colitis, that involve chronic inflammation of the digestive tract. Symptoms of IBD include, without limitation, abdominal pain, diarrhea, rectal bleeding, severe internal cramps, weight loss and anemia.

The phrase “KSL cells” refers to an early form of mouse/murine hematopoietic stem and progenitor cells. Characteristic markers for such cells are Kit (+), Sca-1 (+) and Lin (-). HSCs in mouse show phenotypic markers as being CD34-, CD150+, and Flt3- for LTR (long-term reconstitution). HSCs are identified on the basis of Flt3, Cd48 and Cd150 expression.

The term “MPP3” refers to multipotent progenitors immediately downstream of HSCs. They are primed to differentiate into myeloid cells such as granulocytes and monocytes.

The phrase “Forward versus side scatter”, “FSC vs SSC” or FSC/SCC profile refers to a commonly used method for identifying cells of interest based on size and granularity (complexity)based on light refraction. Scatter can vary depending on the sample, the sheath fluid and the laser wavelength. FSC vs SSC profile is most useful for blood samples.

The phrase “cytokine storm” refers to excessive cytokine release by immune (and other) cells that damages the tissues and organs. In some patients the response is so sever it causes death.

Kit (CD117) is the receptor of Stem Cell Factor. Sca-1 is a murine hematopoietic stem cell antigen. Lin is a series of lineage marker antigens that identify mature murine blood cells.

The phrase “t-distributed stochastic neighbor embedding” or “t-SNE” refers to a statistical method for visualizing high-dimensional data by giving each datapoint a location in a two or three-dimensional map. It is a nonlinear dimensionality reduction technique well-suited for embedding high-dimensional data for visualization in a low-dimensional space of two or three dimensions. Specifically, it models each high-dimensional object by a two- or three-dimensional point in such a way that similar objects are modeled by nearby points and dissimilar objects are modeled by distant points with high probability.

The phrase “ATAC-seq” or “Assay for Transposase-Accessible Chromatin using sequence” refers to a technique used in molecular biology to assess genome-wide chromatin accessibility. In 2013, the technique was first described as an alternative advanced method for MNase-seq, FAIRE-Seq and DNase-Seq. ATAC-seq is a faster and more sensitive analysis of the epigenome than DNase-seq or MNase-seq

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

Down-modulating or inhibitory nucleic acids include, without limitation, antisense molecules, aptamers, ribozymes, triplex forming molecules, RNA interference (RNAi), CRISPR (Clustered Regularly Interspaced Short Palindromic Repeats) RNA (crRNA), and external guide sequences. These nucleic acid molecules can act as effectors, inhibitors, modulators, and stimulators of a specific activity possessed by a target molecule, or the functional nucleic acid molecules can possess a de novo activity independent of any other molecules. In certain embodiments, inhibitory nucleic acids are employed.

Antisense molecules are designed to interact with a target nucleic acid molecule through either canonical or non-canonical base pairing. The interaction of the antisense molecule and the target molecule is designed to promote the destruction of the target molecule through, for example, RNase H mediated RNA-DNA hybrid degradation. Alternatively, the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (Kd) less than or equal to 10-6, 10-8, 10-10, or 10-12. A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10^{-6,} 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426. Gene expression can also be effectively silenced in a highly specific manner through RNA interference (RNAi). This silencing was originally observed with the addition of double stranded RNA (dsRNA) (Fire, A., et al., Nature, 391:806-11 (1998); Napoli, C., et al., Plant Cell, 2:279-89 (1990); Hannon, G. J., Nature, 418:244-51 (2002)). Once dsRNA enters a cell, it is cleaved by an RNase III-like enzyme, Dicer, into double stranded small interfering RNAs (siRNA) 21-23 nucleotides in length that contain 2 nucleotide overhangs on the 3′ ends (Elbashir, S. M., et al., Genes Dev., 15:188-200 (2001); Bernstein, E., et al., Nature, 409:363-6 (2001); Hammond, S. M., et al., Nature, 404:293-6 (2000)). In an ATP-dependent step, the siRNAs become integrated into a multi-subunit protein complex, commonly known as the RNAi induced silencing complex (RISC), which guides the siRNAs to the target RNA sequence (Nykanen, A., et al., Cell, 107:309-21 (2001)). At some point the siRNA duplex unwinds, and it appears that the antisense strand remains bound to RISC and directs degradation of the complementary mRNA sequence by a combination of endo and exonucleases (Martinez, J., et al., Cell, 110:563-74 (2002)). However, the effect of RNAi or siRNA or their use is not limited to any type of mechanism.

Small Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al., Nature, 411:494 498(2001); Ui-Tei, K., et al., FEBS Lett, 479:79-82 (2000)). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion’s SILENCER.RTM. siRNA

The following materials and methods are provided to facilitate the practice of the present invention.

### Mice

Rosa26-CreER^{T2} TKO and CT mice (mixed 129/Bl6 background) were previously described⁶ and Cre expression was induced by intraperitoneal injection of 1 mg of Tamoxifen® (Sigma) in corn oil for five consecutive days. Csf2rb deficient mice were obtain from Jax laboratory (Ref 005940, Bl6 background)). Wild-type Bl6 mice were used as controls for experiments involving Csf2rb deficient mice. At 12-13 weeks of age (age of mice used in inflammatory model assays), average weight for male wild-type Bl6 is ~27-28 gr while average weight for female wild-type Bl6 is ~20-21 gr. Mice were housed in the CHOP barrier facility. Inflammation models were generated by daily injection of Il1β (general inflammation model) and LPS (Lipopolysaccharide, systemic infection model) as well as addition of DSS 2% w/v (Dextran Sulfate Sodium, colitis model) to drinking water. Il1β (Peprotech), LPS and DSS (Sigma) were reconstituted with PBS (Phosphate Buffer Saline). Mice from different genotypes used in these experiments were sex- and age- matched to facilitate comparative analysis. All experiments were approved by CHOP IACUC (protocol #969).

### Flow Cytometry and Transplantation

Cells from the BM were filtered, and red blood cells were lysed with the ACK buffer (NH₄Cl/KHCO₃). The remaining white blood cells were stained with a cocktail of lineage antibodies (Cd3, Cd4, Cd8, B220, Ter119, Mac1 and Gr1) as well as antibodies against Kit, Sca1, Flk2/Flt3, Cd48, Cd150, FcgR, Cd34, Ly6c, Ly6g, Rank and Cd115 (eBiosciences) and βchain receptor (Miltenyi). In addition, white blood cells were further identified based on their FSC (Forward Scatter)/SSC (Side Scatter) profile. For transplantation, two million unfractioned BM cells were collected from CT and Rosa26-CreER^{T2} TKO mice and retro-orbitally transplanted into lethally irradiated (8G) Rag 1^{-/-} mice. Recipient mice were injected with Tamoxifen five weeks after transplantation.

### Colony Assay and Cytospin Analysis

100 CT or TKO HSPC subpopulations were plated in 1 ml of methylcellulose, either pre-supplemented with cytokines (M3434) or not (M3334) (Stem Cells Technologies). Cytokines were obtained from peprotech and resuspended in PBS. CSL311 (Abcam 245696) was added to the methylcellulose media at increasing concentration prior to cell plating. Colonies were counted after 9 days in culture. Colony cells were resuspended, counted and spun for histology analysis. Cytospin were obtained by spinning 20,000 cells at 500 rpm against glass slides. Cells were stained with Giemsa stain, as described previously⁶.

### Ex Vivo Cytokine Stimulation, ChIP and Gene Expression Analysis

HSPCs were flow-isolated from CT and TKO mice two weeks after tamoxifen treatment and serum-starved for two hours prior to stimulation with 10 ng/ml of Il3 and GM-Csf (Peprotech) for 10 minutes. Cells were fixed, permeabilized and stained for anti p-Erk (Cell Signaling) prior to analysis on LSR Fortessa. For ChIP assay, primary BM cells were collected into 0.5% SDS lysis buffer at a density of 50 x 10⁶ cells/mL and crosslinked in 2% formaldehyde for 10 minutes. Lysates were sonicated on a BioLogics Model 3000 Ultrasonic Homogenizer for 30 seconds at 40% power on ice to produce chromatin fragments of approximately 400-700bp. Antibodies against E2f1 and E2f3 were purchased from Santa Cruz. For RT-qPCR, purified hematopoietic populations were sorted in Trizol LS and the extracted RNA was purified with the RNeasy Mini kit (Qiagen), followed by DNAseI digestion to minimize the risk of genomic contamination. Reverse transcription was performed with the Protoscript First Strand cDNA Synthesis kit (New England Biolabs) and qPCR was performed in duplicate with SYBR Green PCR Master Mix (Life Technologies) on the Viia7 Real-Time qPCR system (Life Technologies). Data were normalized using gapdh as a reference gene. Primer sequences are available upon request.

### In Silico ATAC-Seq and 10x Genomics

Sample preparation: ATAC-Seq: ATAC-seq libraries were prepared as described⁵⁷. In brief, 50,000 cells were isolated by flow cytometry and washed with cold 1x PBS. Cell pellet were resuspended in 50 µl of cold lysis buffer (10 mM Tris-HCl, pH 7.4, 10 mM NaCl, 3 mM MgCl₂ and 0.1% (v/v) Igepal CA-630) and immediately applied to centrifugation at 1,600xg, 4° C. for 10 min. Nuclei pellet was resuspended in 50 µl of transposition reaction mix (1x Tagment DNA Buffer, 2.5 µl of Tagment DNA Enzyme 1 (Illumina)) and incubated for 30 min at 37° C. Subsequent steps of the protocol were performed as previously described (Buenrostro et al., 2013). Libraries were purified using a Qiagen MinElute Gel Purification kit and the concentrations were measured using both Qubit and KAPA qPCR. 2100 Bioanalyzer was used to check the quality of libraries. Libraries were sequenced on the Illumina NextSeq 500, with 75-bp paired-end reads. 10x genomics: cells were sorted by flow cytometry and resuspended at a concentration of 1000 cells/ul. Cell suspension were processed by the Next Generation Sequencing Core at Penn Medicine.

Data processing: ATAC-seq: after trimming the adapters with attack (version 0.1.5 -https://atactk.readthedocs.io/en/latest/index.html), the raw reads were aligned to the mm9 genome using bow-tie-1.1.2 ((https://genomebiology.biomedcentral.com/articles/10.1186/gb-2009-10-3-r25) with the following flags: --chunkmbs2000—sam—best—strata-ml-X2000. We used MACS2 (https://www.ncbi.nlm.nih.gov/pubmed/18798982) for peak calling with a q cutoff of 0.05. Downstream analysis and visualization was done using HOMER (http://homer.ucsd.edu/homer/ and deepTools2 (https://www.ncbi.nlm.nih.gov/pubmed/27079975). scRNA-seq: to process the raw data, we used cellranger (v 2.0.0), which includes read alignment and generating a gene by cell count matrix. The R package Seurat (v 2.3.0) (Stuart*, Butler*, et al., Cell 2019) was utilized for filtering out cells with high mitochondrial signal, clustering and visualization. Furthermore, single-cell trajectories were identified and examined using Monocle 2 (v 2.10.1) (PMID: 28825705) and Monocle 3 (v 3_0.2.0) (PMID: 30787437). Finally, we reconstructed gene regulatory using the SCENIC (v 1.1.2-2) (PMID: 28991892) package.

### P-Sicor-RFP Lentiviral Vectors

The pSicoR-RFP lentiviral vector was generously provided by the Ventura lab (available on the World Wide Web at mskcc.org/research/ski/labs/andrea-ventura). 293 cells were transfected with pSicoR-RFP vectors expressing either a scramble hairpin or two hairpins (a and b) targeted against genes of interest (Csf2rb and Irf8) as well as envelope plasmids (VSV-G: available on the World Wide Web at addgene.org/8454/; DR8.2: https://www.addgene.org/8455/). Supernatant of 293 cells was collected 48 hours after transfection and spun with target cells and polybrene (10ug/ml) for 2 hours at 2,000 rpm for lentiviral transduction. Successfully transduced RFP+ target cells were isolated by flow cytometry 48 hours after infection.

### METHODS AND COMPOSITIONS

We describe a novel molecular mechanism that enhances the production of myeloid cells from MPPs in response to hematopoietic stress. In particular, we identify the transactivation of Csf2rb by E2f, and the subsequent enhancement of βcytokines signaling, as a critical mechanism for expanding the MPP4 subpopulation and accelerating its differentiation through myelopoiesis. An object of the invention is to block this enhancement of βcytokine signaling to repress the formation of myeloid cells.

Cellular division is a process that requires the integration of multiple biological activities such as cell cycle, metabolic changes, cell fate decision, motility, etc. Myeloid cells are short-lived and hematopoietic stress requires a burst of fresh myeloid cells to address the source of stress. E2f is predominantly studied for its critical role in the regulation of cell cycle. However, limited evidence suggests that E2f is also involved in other aspects of cell division, such as the regulation of glucose metabolism, self-renewal decision, and progenitor cell fate decision. Here, we show that E2f coordinates the proliferation and the enforced differentiation of the lymphoid-primed MPP4 towards the myeloid lineage. From an evolutionary standpoint, establishing a central factor to ensure that bipotential MPP4, the most abundant HSPC subpopulations, overwhelmingly differentiates into myeloid cells increases the efficiency of HSPC response to the stress that triggers their proliferation.

Merged sc-Seq analysis of distinct hematopoietic progenitor populations such as MPP4 and MP subpopulations reveals little overlapping in control conditions, indicating that the differentiation of lymphoid-primed MPP4 cells into committed MP cells is not a continuum but rather a marked alteration of their identity. In contrast, there is a significant overlap between the distribution of TKO MPP4 and TKO MP cells, indicating that the differentiation of TKO MPP4 cells into MP cells is accelerated. In particular, sc-Seq gene expression and trajectory analysis of CT and TKO MPP4 shows that TKO MPP4 cluster 2 expresses a myeloid signature at much higher levels compared to CT MPP4. Finally, the myeloid-biased differentiation of TKO MPP4 involves limited chromatin remodeling, suggesting that the process that alters MPP4 cell differentiation in response to hematopoietic stress is very reactive and requires little structural changes of the chromatin landscape. Collectively, our data indicates that proliferation is associated with a biased as well as accelerated differentiation of HSPCs through the myeloid lineage.

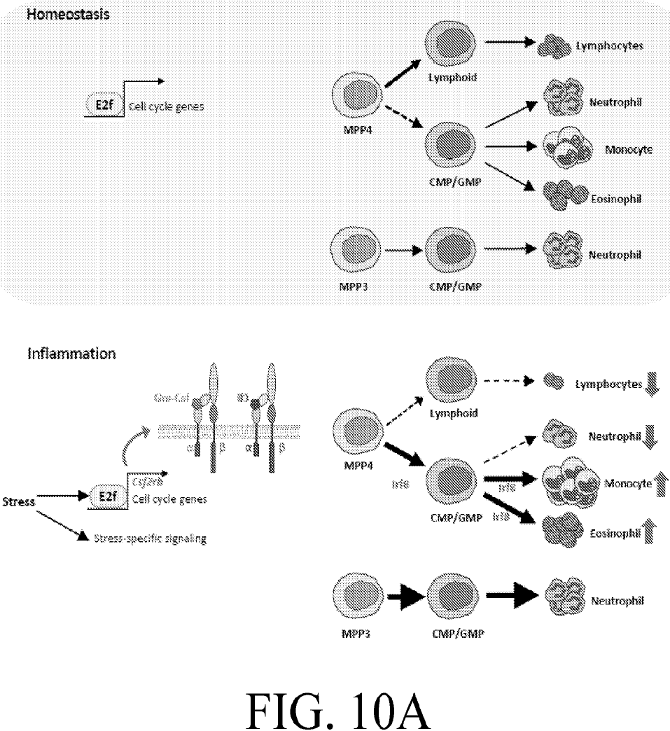

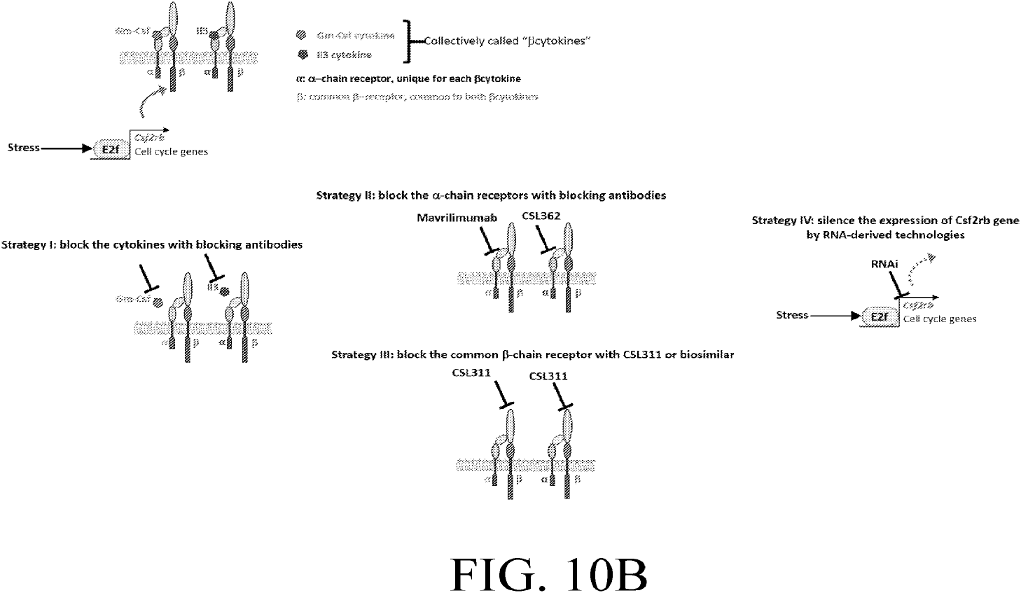

The cell autonomous inactivation of negative regulators of cell cycle is sufficient to enforce the myeloid differentiation of proliferative HSPCs. Our results identify the cell intrinsic mechanism that connects proliferation and myeloid-biased differentiation of MPP4 cells. In particular, we find that E2f coordinates the activation of a cell cycle program with the transactivation of Cs2rb in HSPCs and their myeloid progeny. In MPP4, the resulting enhanced βcytokine signaling transactivates Irƒ8 to drive MPP4 differentiation towards the monocyte lineage. In addition, we find that enhanced βcytokine signaling increase the cellular output of myeloid-primed MPP3. In this cell-intrinsic model, the role of the extrinsic stress is two-fold: 1) trigger MPP4 proliferation by disrupting the Rb/E2f interaction; 2) exacerbate undesirable intracellular signaling (initiated for example by other cytokine signaling) to the enhanced βcytokine signaling activated by E2f to influence the production of specific myeloid subpopulations (FIG. 10). This multi-layer mechanism for cell fate decision explains the variability in the identity of the myeloid subpopulation(s) produced, based on the nature of the stress.

Although an appropriate HSPC response is required in the context of a hematopoietic stress, exposure to a severe stress can lead to organ failure. In addition, exposure to a chronic stress can progressively exhausts HSPCs and lead to the unnecessary accumulation of myeloid cells, resulting in continued inflammation and organ damage. Our data, showing that targeting βcytokine activity decreases the response of HSPC and their progeny to hematopoietic stresses, indicates that βcytokines represent a therapeutic target to treat patients suffering for the consequences of severe and/or chronic exposure to multiple forms of hematopoietic stress. In this context, a pan-βcytokine inhibitor, such as CSL311® should be effective. Our data shows that other compounds, such as CSL362, and Mavrilimumab that target individual family members such as GM-CSF and Il3 can be also be effective when used in combination to modulate the consequences of severe and chronic hematopoietic stressors, including for example, infection, inflammation, aging, hypercholesterolemia, myocardial infarction, ischemia, and social stress. These conditions can trigger undesirable increased proliferation and biased differentiation of HSPCs towards specific mature myeloid subpopulation(s).

The following examples are provided to illustrate certain embodiments of the present invention. They are not intended to limit the invention in any way.

### Example 1 Rb Family Inactivation Triggers Monocytosis

We previously reported that inactivation of the Rb family (TKO) triggers HSPC exit from quiescence and the development of myeloproliferation characterized by increased frequency of monocytes/macrophages and eosinophils⁶. Analysis of the Mac1+ (an ubiquitous myeloid marker) population from CT (Rb^{lox/lox}, p130^{lox/lox} p107^{-/-}) and TKO (Rosa26-CreER^{T2} Rb^{lox/lox}, p130^{lox/lox} p107^{-/-}, where Cre activity is triggered upon intraperitoneal Tamoxifen injection) bone marrow (BM) with Ly6c (monocyte marker) and Ly6g (granulocyte marker) confirms the expansion of Ly6c+/Ly6g- monocytes (FIGS. 1A-1B). Downstream of monocytes, analysis of Rank expression identifies the expansion of Rank+ osteoclasts (FIG. 1B) in TKO mice, confirming the expansion of the entire monocyte lineage upon inactivation of Rb family. Correlating with increased osteoclast frequency, analysis of control and TKO bone structures reveal increased osteoclastic activity and impaired bone structure in TKO mice (FIG. 1C). Collectively, these results suggest that Rb family inactivation coordinates HSPC exit from quiescence with stress monocytosis

### Example 2 Rb Family Inactivation Impairs LT-HSC Self-Renewal Without Skewing Their Differentiation

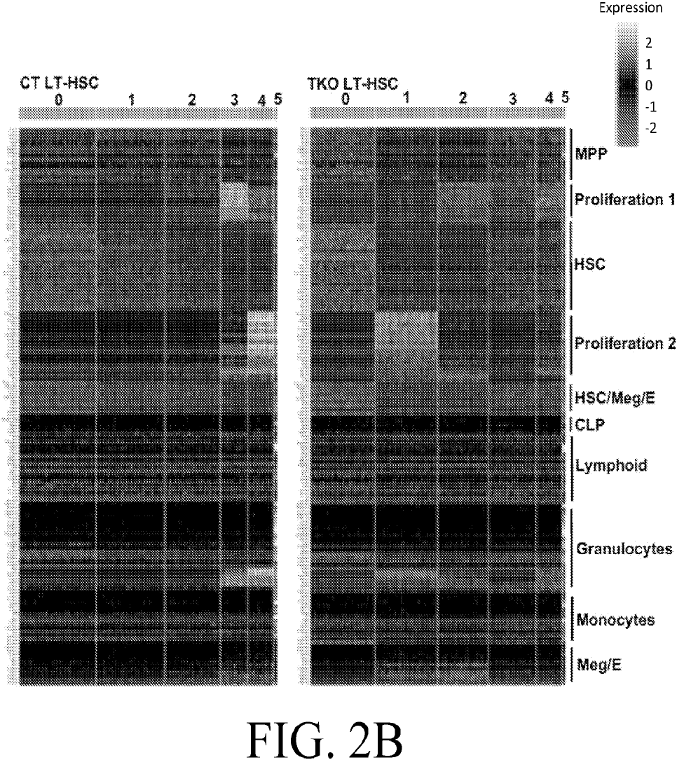

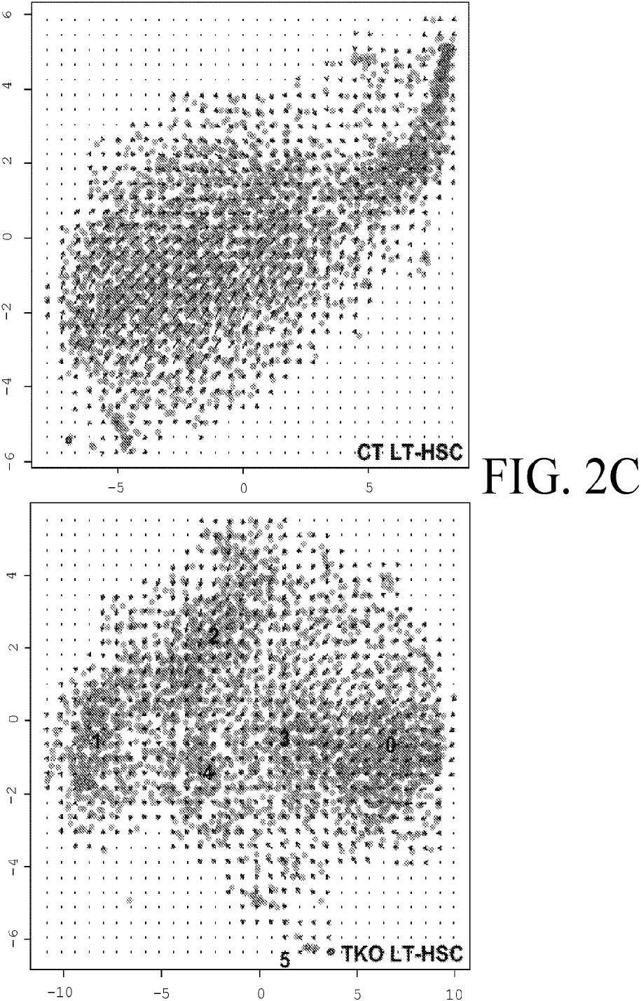

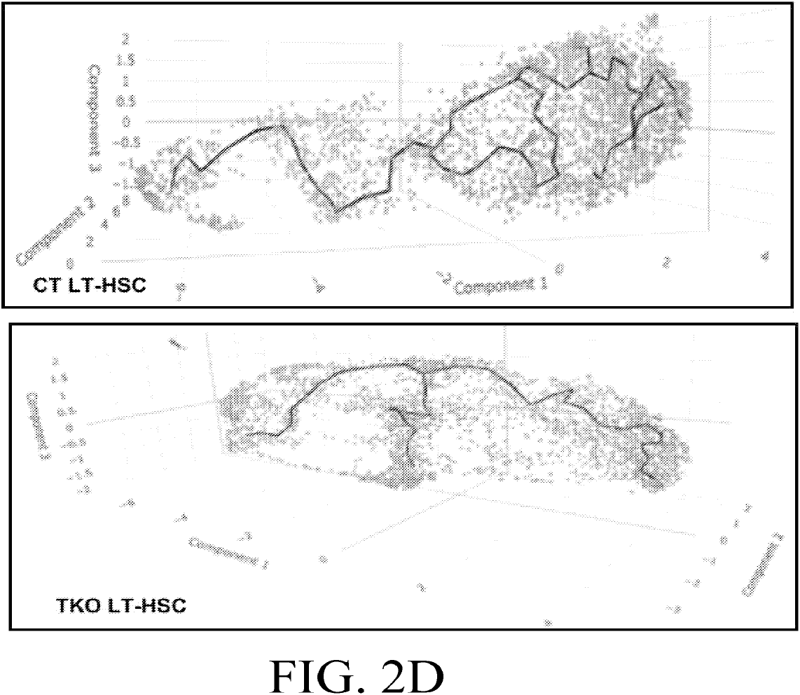

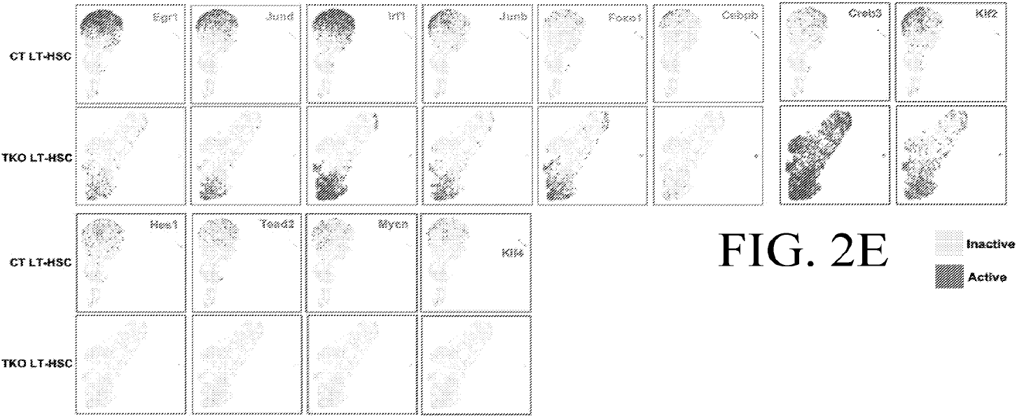

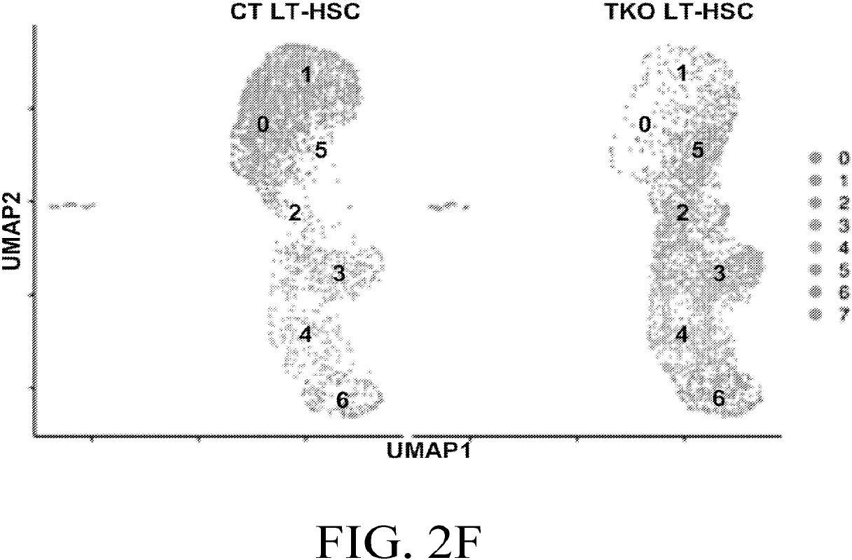

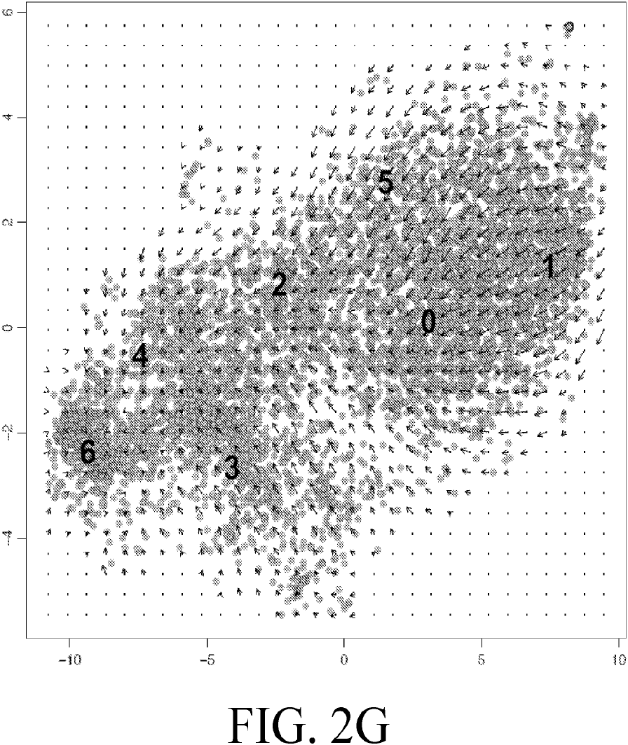

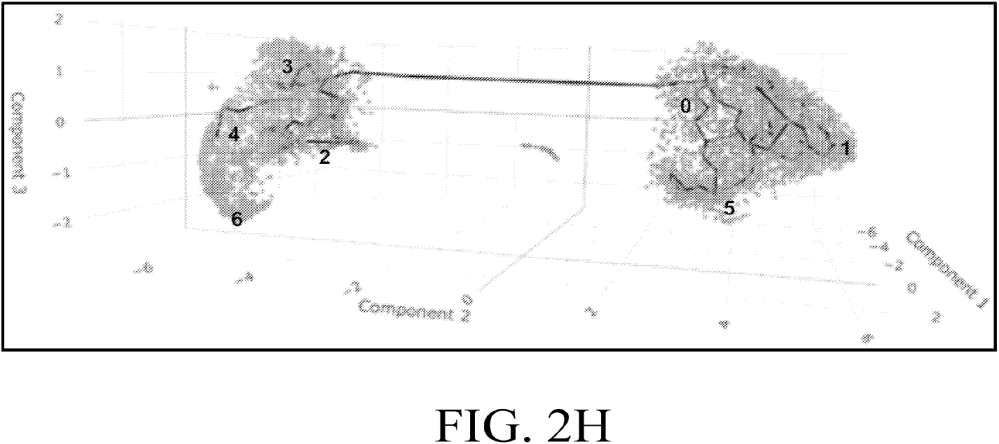

To determine the mechanisms driving stress monocytosis upon HSPC exit from quiescence, we first performed 10x single-cell RNA-Sequencing (scRNA-Seq) on LT-HSCs isolated from the BM of CT and TKO mice two weeks after Rb family inactivation. Seurat analysis identifies 5 clusters in each genotype, organized in a linear fashion (FIG. 2A). Gene expression analysis shows that a stem cell transcriptional signature is associated with the most quiescent clusters in CT LT-HSCs (clusters 0-2). In contrast, an erythro-myeloid transcriptional signature is associated with features of proliferation in clusters 3 and 4 (FIG. 2B). Inactivation of Rb family genes does not significantly alter the transition from a stem cell to an erythro-myeloid signature (FIG. 2B), although the frequency of cells expressing erythromyeloid genes (found in TKO clusters 1, 2 and 4) is expanded compared to controls (found in CT clusters 3 and 4). Comparative analysis of the single most primitive cluster from each genotype (cluster 0) shows the earlier onset of a MPP program in TKO LT-HSCs compared to controls. Trajectory analysis reveals that CT LT-HSCs are organized in a linear hierarchy with little overlapping between clusters. In contrast, TKO LT-HSC clusters are organized in two branches (2 and 0/3/4) with more pronounced overlap, which ultimately progress to cluster 1(FIGS. 2C -2D). Therefore, inactivation of Rb family activates proliferation in LT-HSCs and disrupts their linear hierarchy.

To determine the transcriptional changes occurring in LT-HSCs upon Rb family inactivation, we performed a SCENIC⁴⁰ analysis of scRNA-Seq data. This approach identifies a series of transcriptional networks that are specifically active in the most primitive CT LT-HSC fraction. Although the activity of most of these networks is conserved in TKO LT-HSCs, Rb family inactivation leads to the loss of several networks that are critical for stem cell maintenance, such as Notch, Yap, Myc and Klf4 (FIG. 2E), further supporting the concept that Rb family inactivation impairs the self-renewal capacity of primitive LT-HSCs.

To further characterize the consequences of Rb family inactivation for LT-HSC biology, we integrated the dataset from both CT and TKO LT-HSCs into a single analysis. This approach shows that downstream clusters (3-5) are shared between both genotypes. In contrast, we found little overlapping between upstream clusters from CT (clusters 0&1) and TKO (cluster 2&5) (FIG. 2F). However, RNA Velocity and Monocles-3D trajectory analysis of the merged datasets show that cluster 2&5 are not located directly downstream of their control equivalents (0&1) within the trajectory but are instead located on the side of the trajectory, suggesting that they represent alternate states that do not typically exist in homeostatic conditions (FIGS. 2G-2H). Collectively, these results show that Rb family inactivation disrupts the quiescence and self-renewal maintenance of the most primitive LT-HSCs. However, they do not show substantial evidence of accelerated differentiation or lineage skewing in TKO LT-HSCs, suggesting that the mechanism that skews differentiation towards monocytosis in proliferative HSPCs occurs in downstream populations.

### Example 3 Rb Family Inactivation Increases MPP4 Frequency and Directs Their Differentiation Towards Myelopoiesis

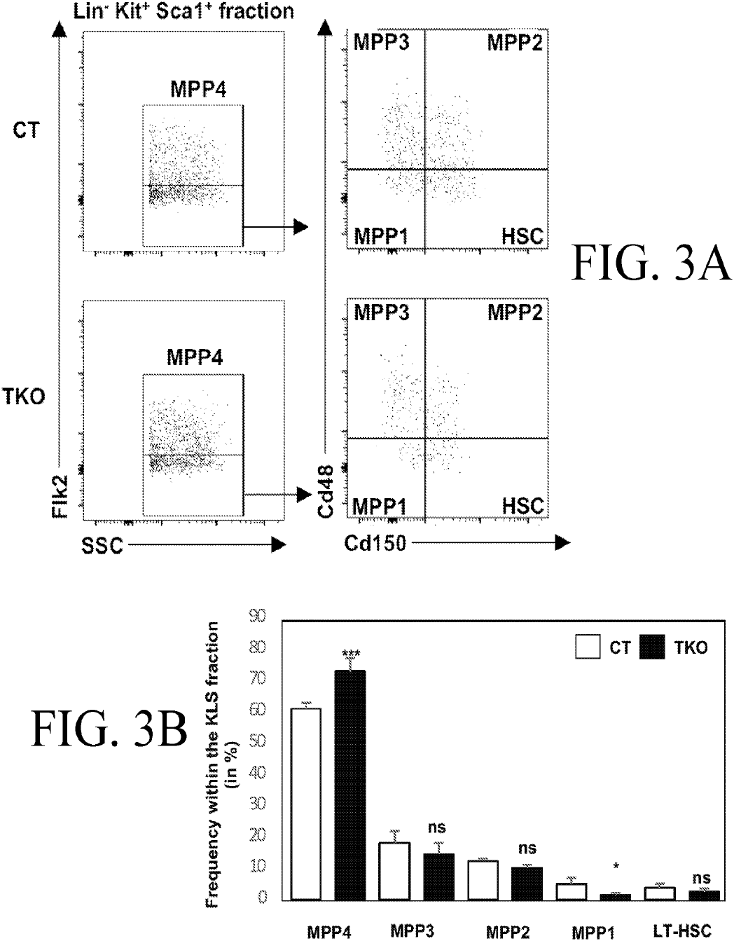

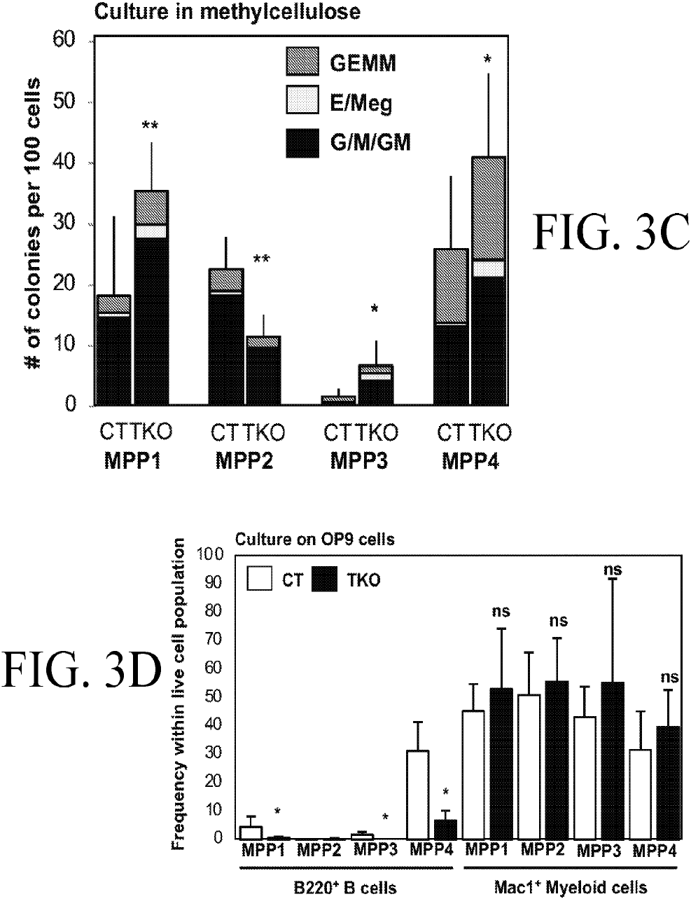

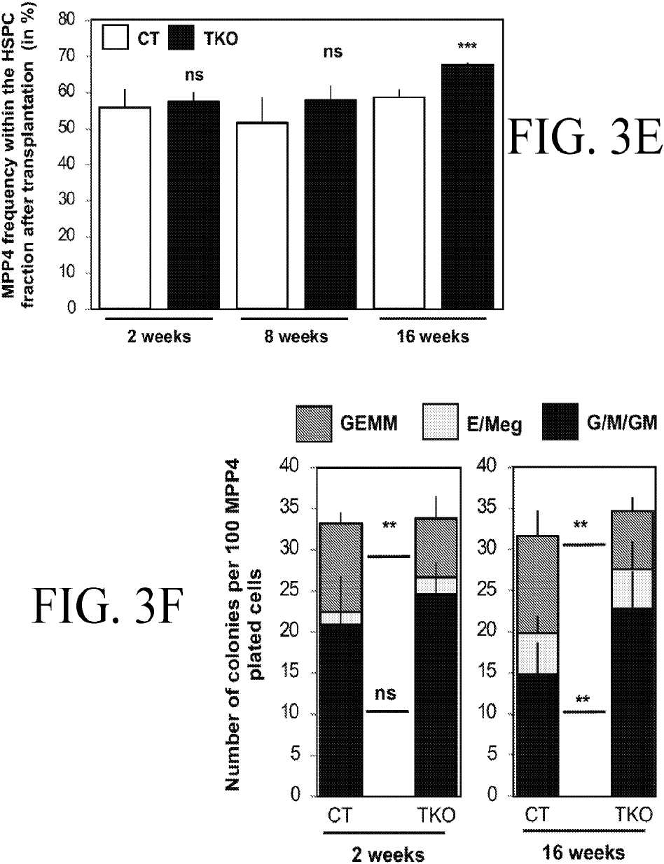

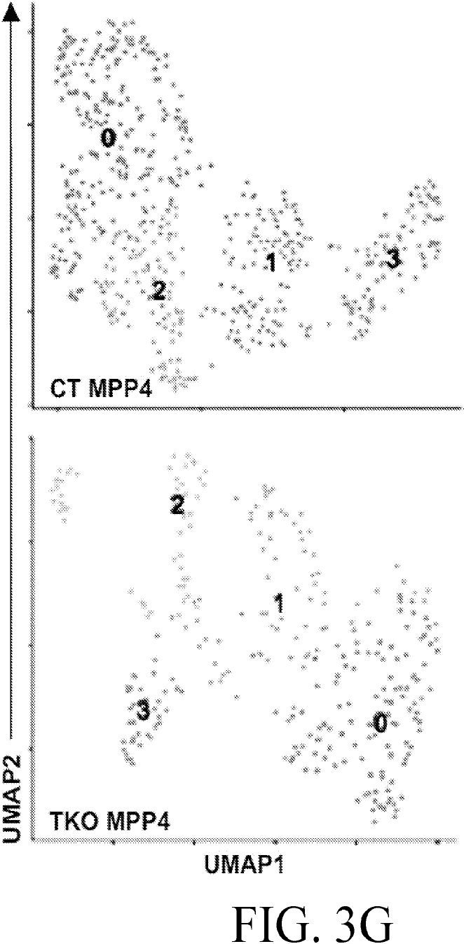

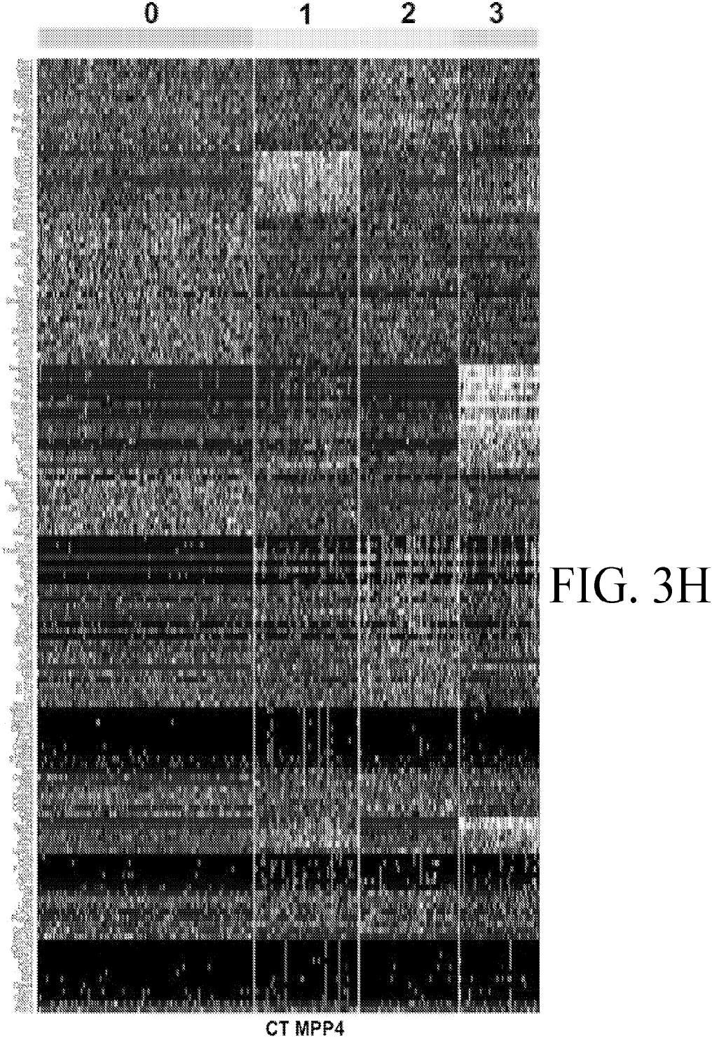

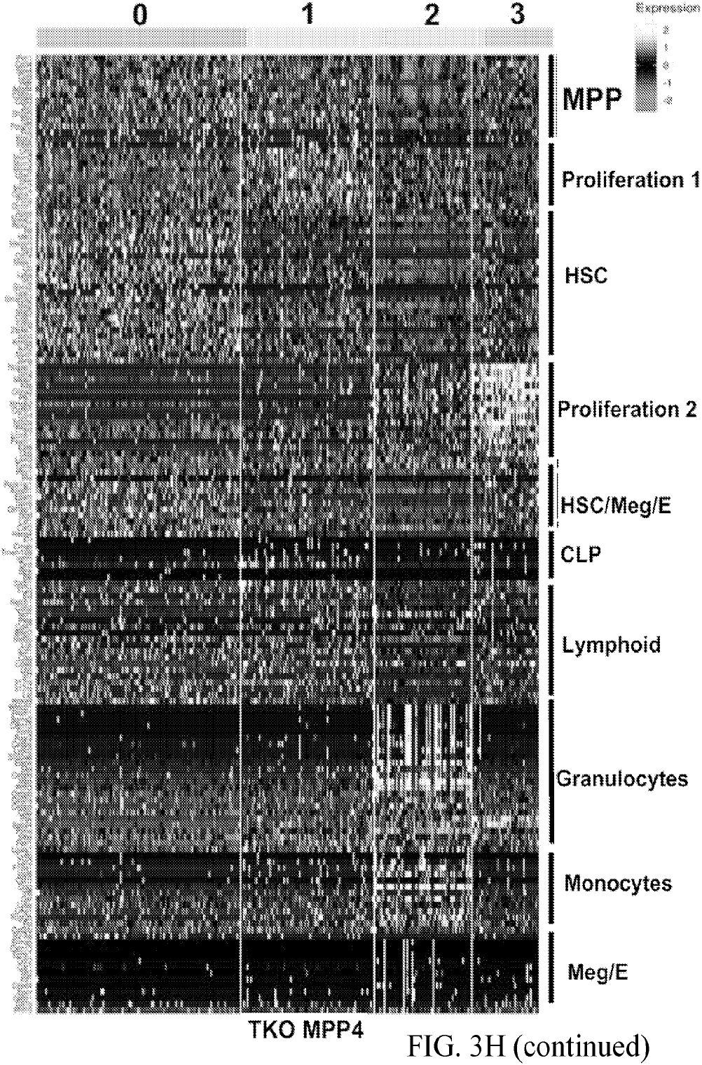

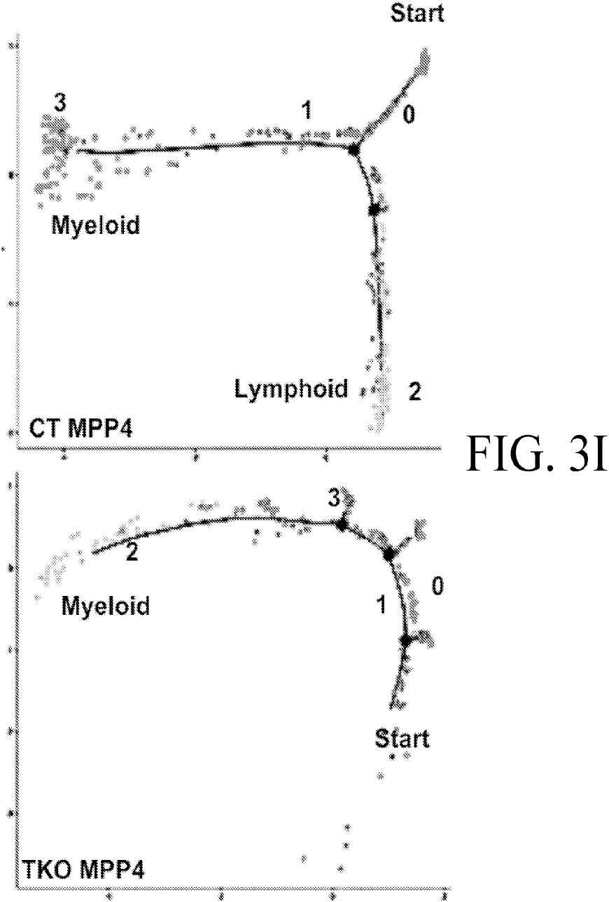

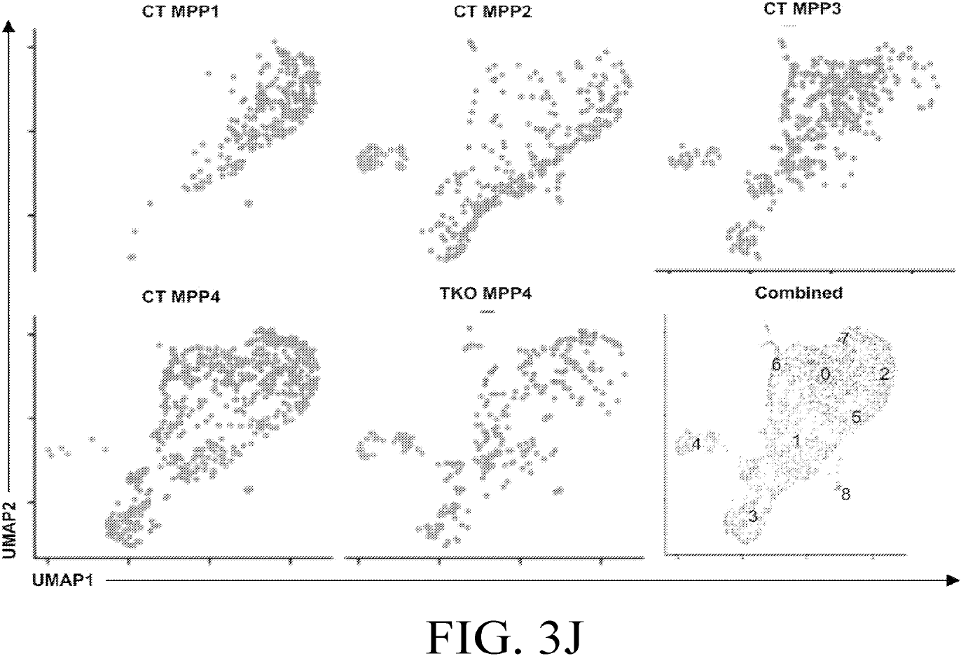

MPP subpopulations are important drivers of hematopoiesis in hemostatic conditions⁴¹ and we sought to investigate whether Rb family inactivation alters their frequency or contribution to hematopoiesis, including monocytosis. Two weeks after Tamoxifen treatment, TKO MPP4 cells are increased in frequency compared to CT MPP4. In contrast, TKO MPP1 frequency is reduced ~2-fold while the frequency of other HSPC subpopulations is unchanged compared to their respective controls (FIGS. 3A-3B). To determine whether Rb family inactivation affects the contribution of lineage-primed MPPs to hematopoiesis, we performed ex vivo culture with MPP1-4 cells isolated from CT and TKO mice two weeks after Tamoxifen treatment. TKO MPP1, 3, and 4 had increased colony-forming activity for whereas TKO MPP2 had decreased colony-forming activity, compared to their respective controls (FIG. 3C). In addition, MPPs were co-cultured with OP9 cells to determine their contribution to lymphopoiesis. While CT and TKO MPP1-3 cells produce marginal amounts of lymphoid cells, TKO MPP4 cells had a decreased capacity to generate lymphoid cells, compared to CT MPP4 (FIG. 3D). Finally, we transplanted Rag1^{-/-} immunodeficient mice with CT or cTKO unfractioned bone marrow (BM) cells, followed by the treatment of recipient mice with Tamoxifen five weeks after transplantation, when donor hematopoiesis is established. In this context, immunophenotypic BM analysis of chimeric mice at different time points shows an accumulation of myeloid cells^{5,}⁶ and a progressive increase of TKO MPP4 frequency compared to CT MPP4 (FIG. 3E). In addition, colony forming activity assay shows an increased frequency of granulocyte/monocyte (G/M/GM) colonies from TKO MPP4 compared to CT MPP4, suggestive of their biased differentiation towards specific myeloid lineages (FIG. 3F). Collectively, these assays show that the frequency of MPP4 increases and their contribution to hematopoiesis shifts towards myelopoiesis upon Rb family inactivation.

To determine the consequences of Rb family inactivation on the fate of individual MPP4 cells, we performed scRNA-Seq analysis of CT and TKO MPP4 isolated from the corresponding mice two weeks after Tamoxifen treatment. Seurat analysis identified 4 clusters in each population, although their spatial distribution varies between CT and TKO MPP4 (FIG. 3G). Gene expression is overall conserved in the respective clusters 0, 1 and 3 from CT and TKO MPP4. In contrast, differential gene expression programs drive the observed spatial separation between cluster 2 in CT and TKO MPP4 cells. Whereas cluster 2 from CT MPP4 cells enrich for quiescent and lymphoid cell signatures, cluster 2 from TKO MPP4 cells enrich for proliferation and granulocyte/monocyte signatures, suggestive of a strong bias toward myeloid commitment (FIG. 3H). Trajectory analysis of CT MPP4 cells shows that the more primitive cluster 0 differentiates into both lymphoid (cluster 2) and myeloid (cluster 1 and 3) lineages. In contrast, cluster 0 exclusively differentiates into the myeloid lineage in TKO MPP4. In particular, the trajectory of cells in cluster 2 is exaggerated beyond cluster 3, suggesting an exclusive and accelerated differentiation towards myelopoiesis in TKO MPP4 (FIG. 3I).

Next, we performed additional scRNA-Seq for individual CT MPP1-3 subpopulations. Merging of individual sc-Seq data for CT MPP1-4 and TKO MPP4 identified 9 different clusters with various degrees of lineage commitment (FIGS. 3J-K). Based on both the expression of MPP and HSC signatures as well as trajectory analysis, CT MPP4 population displays a high frequency of cells within the most primitive clusters (cluster 2, 0 and 5) as well as cells with a lymphoid signature (cluster 6). On the other hand, TKO MPP4 cells display a lower frequency of cells within the most primitive clusters and a near absence of cells with a lymphoid signature. Instead, TKO MPP4 display an increased frequency of cells with a myeloid signature (cluster 4), as observed in CT MPP2 and MPP3. Therefore, Rb family inactivation in MPP4 subpopulation alters its cellular composition to adopt a profile related to CT MPP2 and MPP3. Together, these data show that Rb family inactivation expands MPP4 cells and biases their differentiation towards the myeloid lineage.

### Example 4 Rb Family Inactivation Accelerates the Differentiation of MPP4 Towards Myelopoiesis

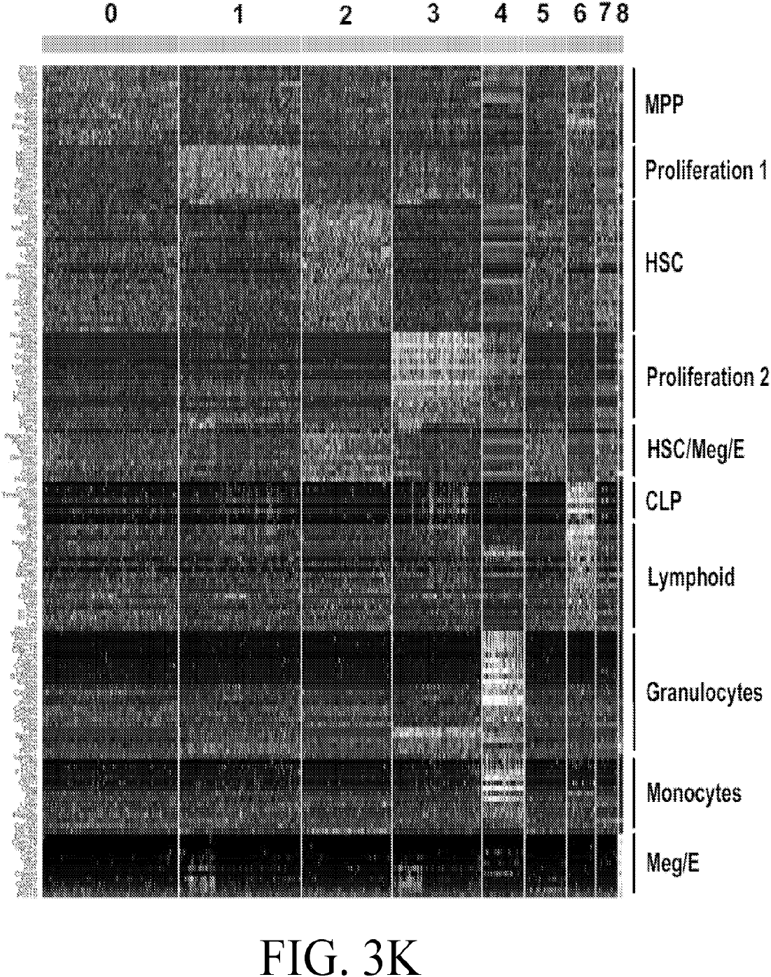

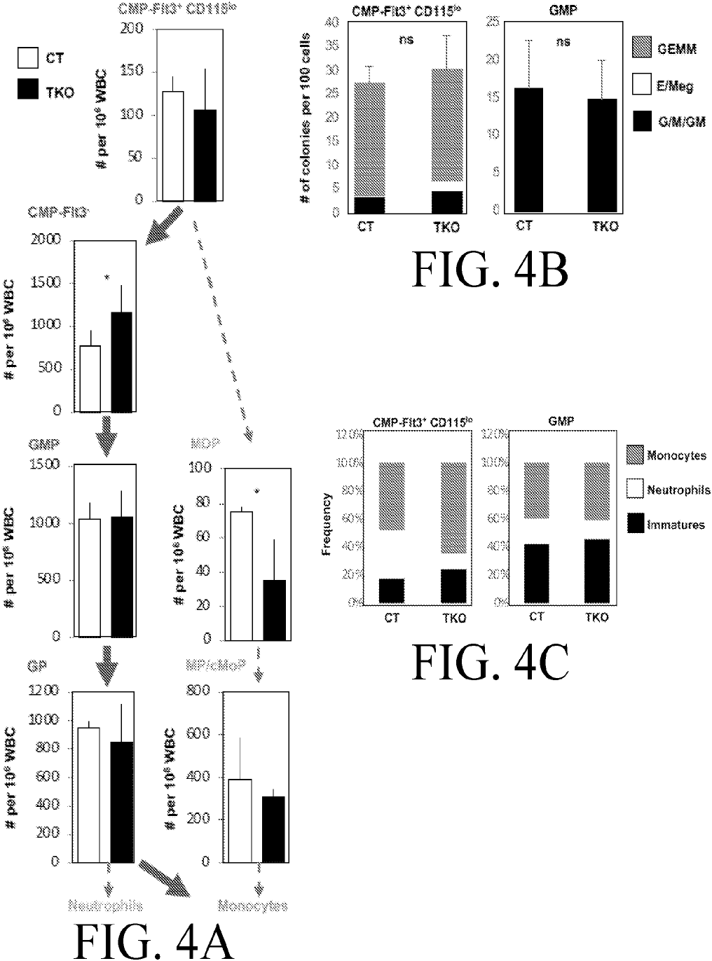

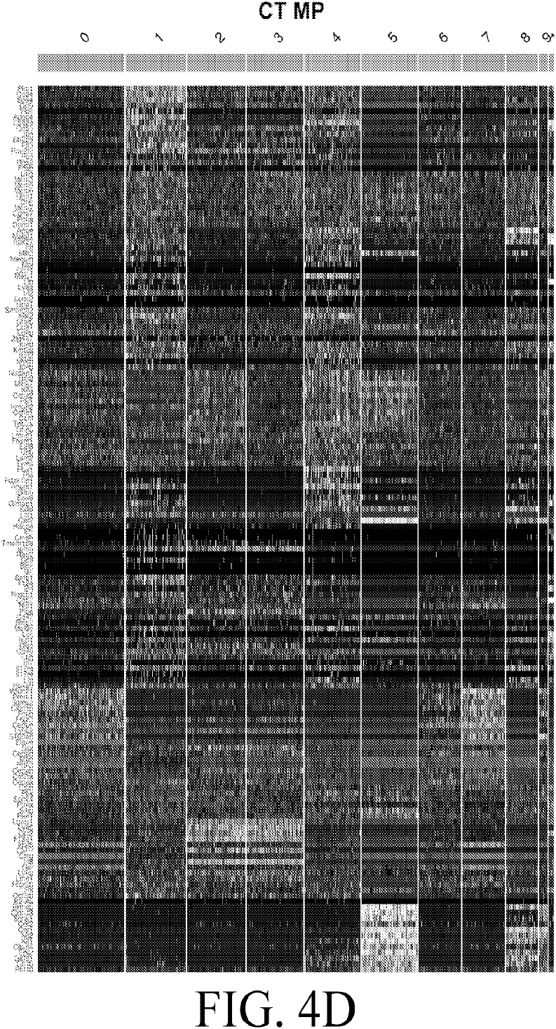

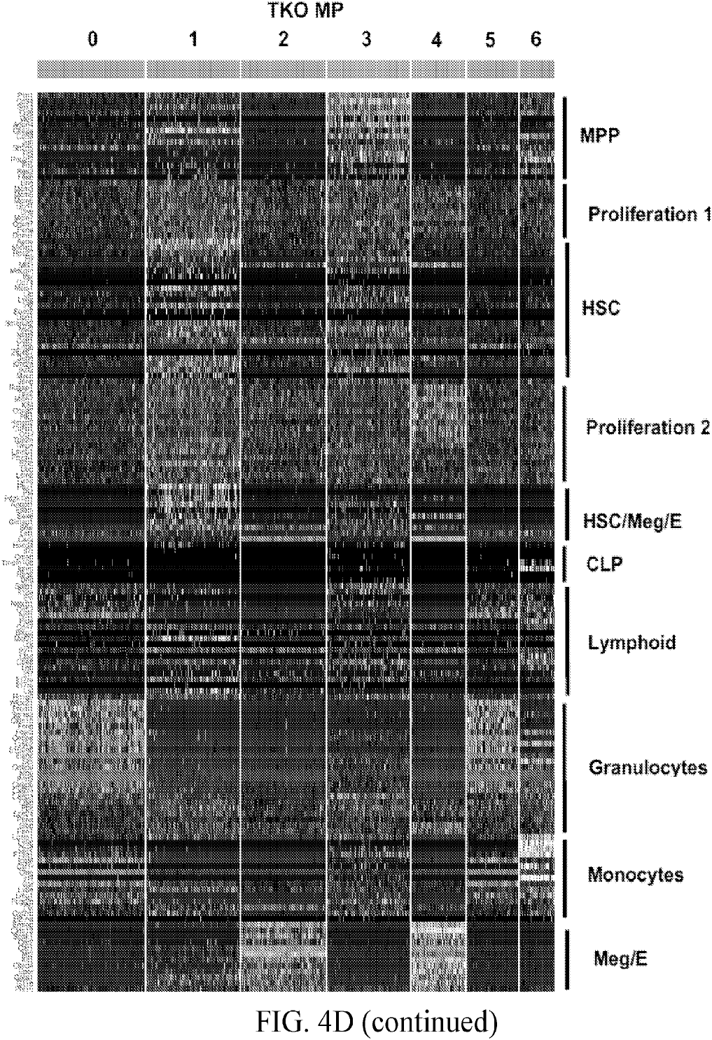

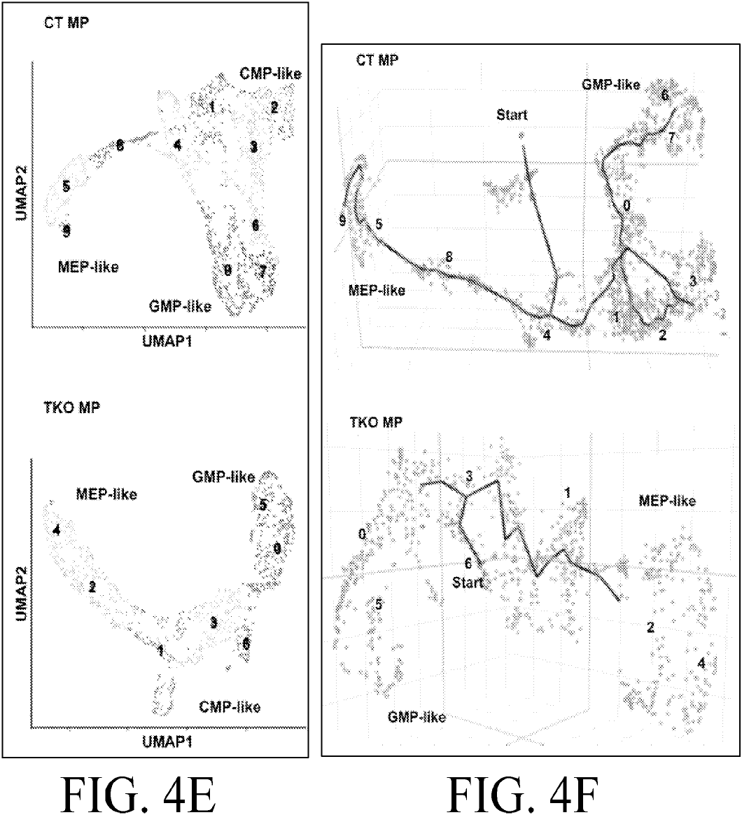

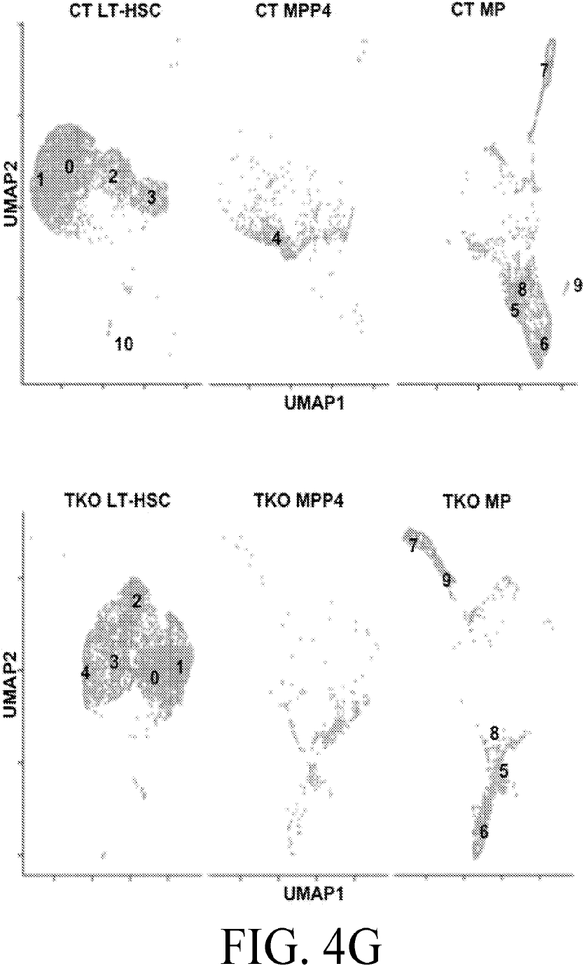

MPPs differentiate into lineage-committed myeloid progenitors (MPs, found in the Lin⁻ Kit⁺ Sca1⁻ fraction), which consist of an ensemble of distinct subpopulations with progressive lineage commitments that fuel both erythroid/megakaryocyte and myeloid lineages^{42,43}. We initially reported that MPs expand upon inactivation of Rb family, in contrast to Common Lymphoid Progenitors (CLPs)⁶. In particular, we found that the expansion is restricted to the Granulocyte. Monocyte Progenitor (GMP) population, while the more primitive common myeloid progenitor (CMP) population and the Megakaryocyte/Erythrocyte (MEP) population decrease⁶. Here, we first took advantage of a recently identified combination of surface markers that segregates distinct subpopulations within the CMP/GMP differentiation axis⁴³ to reveal a differentiation bias along this axis: increased frequency of CMP-Flt3⁻ cells and decreased frequency of MDPs (monocyte/dendritic progenitors) upon Rb family inactivation identifies a first biased cell fate decision in the upstream TKO CMP-Flt3⁺ Cd115^{lo} subpopulation. In addition, the increased presence of monocytes and decreased presence of neutrophils upon Rb family inactivation⁶ indirectly suggests a second biased cell fate decision in the downstream GMP population (FIG. 4A). To determine whether Rb family inactivation alters the contribution of CMP-Flt3⁺ Cd115^{lo} and GMP subpopulations to myelopoiesis, we performed ex vivo methylcellulose culture. Rb family inactivation did not alter the number of colonies formed in culture (FIG. 4B). However, TKO CMP-Flt3⁺ Cd115^{lo}, but not GMP, cells displayed increased differentiation into monocytes versus neutrophils compared to their controls, indicating that a biased cell fate decision occurs in primitive MP subpopulations upon Rb family inactivation (FIG. 4C).

Recent data suggest that the MP compartment can be further fractioned into a series of subpopulations that represent discrete intermediary steps towards the generation of terminally differentiated myeloid cells⁴⁴. To investigate the composition of the MP upon Rb family inactivation, we performed scRNA-Seq on CT and TKO MP cells isolated two weeks after Tamoxifen treatment. Seurat analysis identified a distinct cellular distribution of cell populations between CT and TKO MPs. (FIG. 4D and FIG. 4E). In particular, the number of clusters with a myeloid-like signature is decreased from 5 clusters in CT MPs (clusters 0, 2, 3, 6 and 7) to three clusters (clusters 0, 5 and 6) that are more similar to each other (represented as a smaller distance in the UMAP plot) in TKO MPs, suggesting an accelerated differentiation along the myeloid pathway upon Rb family inactivation. Accordingly, trajectory analysis shows that the differentiation of CT MPs includes a short linear path from the most primitive cluster (cluster 4) to MEP-like cells as well as a long and circumvolved path to GMP-like cells (FIG. 4F). In contrast, the opposite trajectories are observed in TKO MPs, with a short linear path from the most primitive cluster (cluster 6) to GMP-like and a longer circumvolved path to MEP-like cells. These data show that Rb family inactivation inverses the differentiation paths of early MP subpopulations to accelerate myelopoiesis.

To gain a more comprehensive understanding of the cellular mechanisms that direct stress myelopoiesis in the context of Rb family inactivation, we performed further analysis on merged datasets for LT-HSC, MPP4 and MP from the same genotype. This analysis shows that CT MPP4 cells partially overlap with upstream CT LT-HSC while displaying limited overlap with downstream CT MPs. This pattern suggests that, although CT MPP4 represent a distinct entity, their identity is closer to upstream primitive populations. However, this pattern is reversed in TKO hematopoiesis, as TKO MPP4 cells show higher overlapping with downstream TKO MPs than upstream TKO LT-HSCs (FIG. 4G). Collectively, these data show that Rb family inactivation accelerates the differentiation of MPP4 and MP cells throughout myelopoiesis.

### Example 5 E2f-Mediated Activation of Csf2rb Enhances Pcytokine Signaling in TKO MPP4

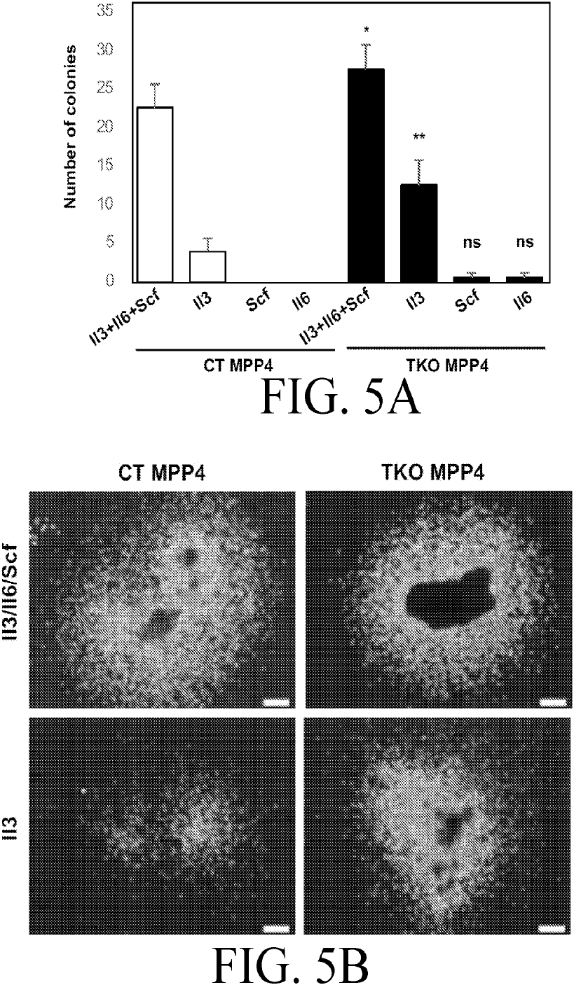

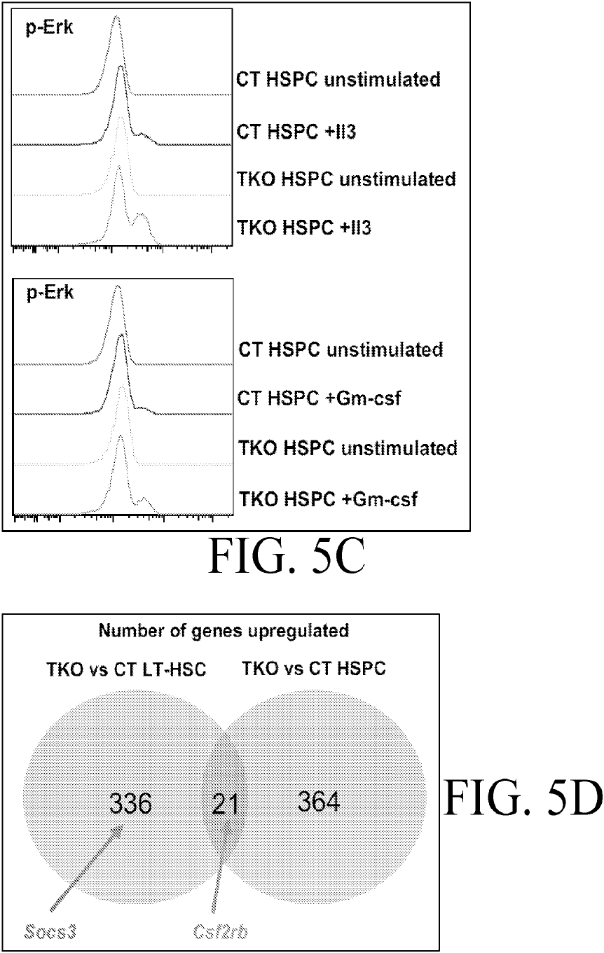

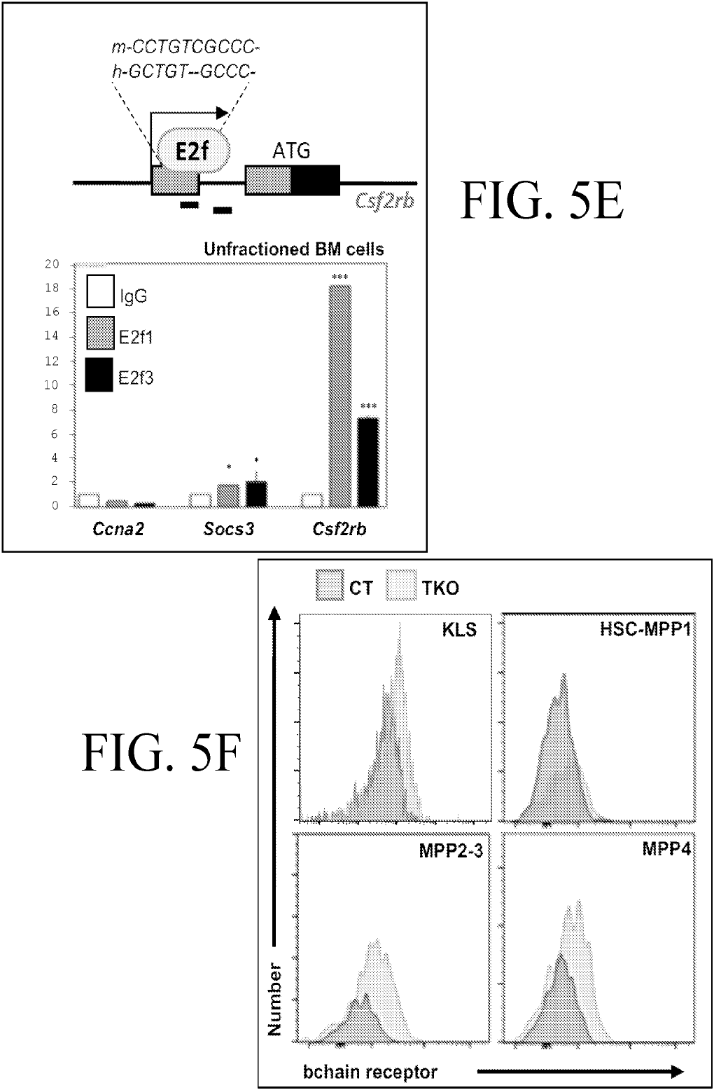

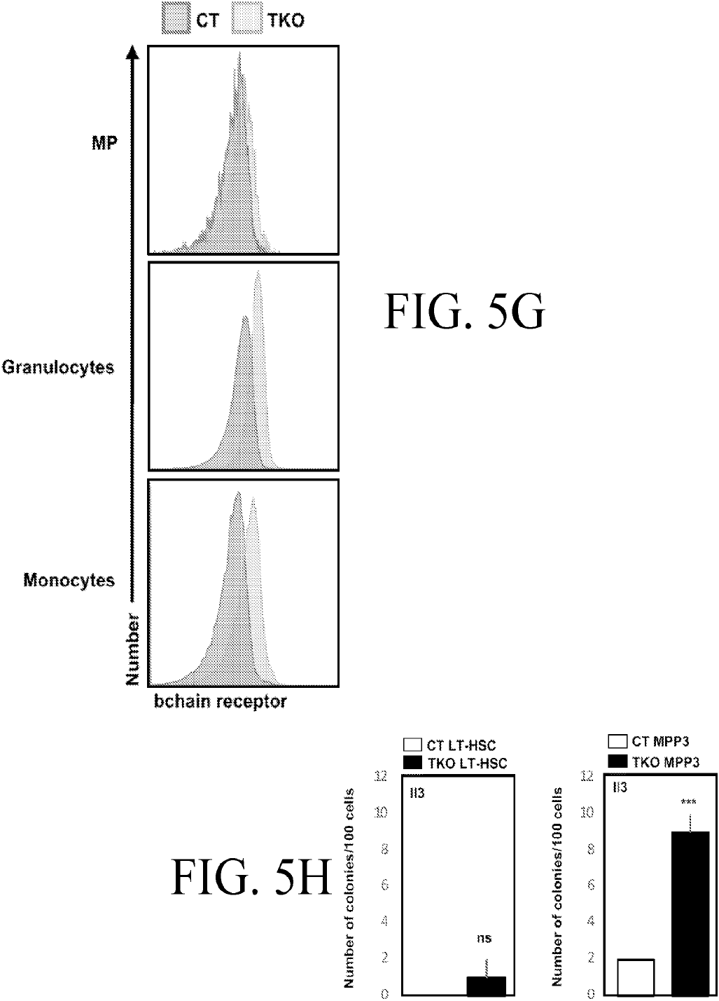

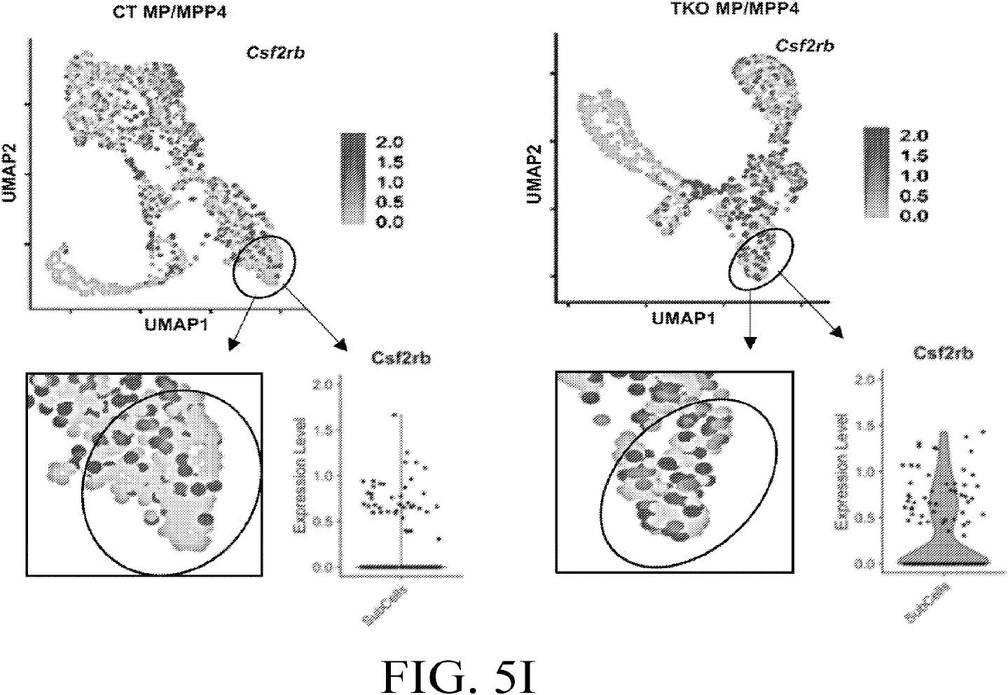

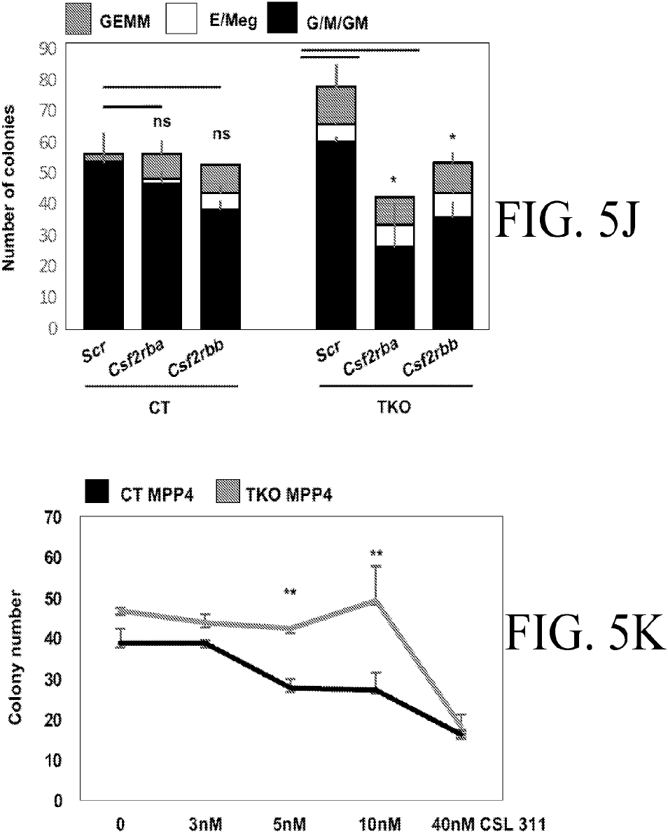

TKO MPP4 do not form colonies ex vivo in the absence of cytokines (not shown), indicating that Rb family inactivation does not confer growth independence. Therefore, we reasoned that TKO MPP4 enhanced colony forming activity (FIG. 3C) may occur in response to cytokine(s) present in the culture media (Il3, Il6, and Scf). To test this hypothesis, we repeated colony assays using methylcellulose media supplemented with Il3, Il6 or Scf, alone or in combination. Neither CT and TKO MPP4 cells exhibit robust colony forming activity in the sole presence of Il6 or Scf. However, while both CT and TKO MPP4 cells form colonies in the presence of Il3 alone, TKO MPP4 display increased colony-forming activity in response to Il3 stimulation compared to CT MPP4 (FIG. 5A). In addition, colonies formed by TKO MPP4 cells in the presence of Il3 are larger compared to colonies formed by CT MPP4 cells (FIG. 5B). Il3, Gm-Csf, and Il5 are members of the βcytokine family, which plays a critical role during in allergic reactions through the promotion of monocyte, dendritic cell and eosinophil lineages⁴⁵⁻⁴⁷. We sought to determine whether enhanced signaling activity in TKO hematopoietic progenitors is specific to Il3 signaling or a common feature of βcytokines. An intracellular phospho-flow (p-flow) assay following stimulation of CT/TKO HSPCs with Il3 or Gm-Csf (the receptor for Il5, Il5Rα, is only expressed in eosinophils) showed increased phosphorylation of Erk (a downstream effector of βcytokine signaling, which also include Stat and Nf-κB factors⁴⁶) in TKO HSPCs compared to CT HSPCs , indicating that enhanced signaling activity is a shared feature of βcytokines in TKO HSPCs (FIG. 5C). However, both CT and TKO MPP4 cells did not grow in the sole presence of Gm-Csf, indicating a dominant function for Il3 at that particular stage of hematopoiesis. βcytokines have unique α-subunit receptors but share a common βsubunit receptor, encoded by Csf2rb, that transmits βcytokine signaling to downstream intracellular cascades⁴⁵⁻⁴⁷. Analysis of our previously published expression data^{5,6} reveals the increased abundance of Csf2rb transcripts in TKO LT-HSCs and the more inclusive TKO HSPC population, compared to CT counterparts (FIG. 5D). Analysis of mouse and human Csf2rb promoter region identifies an evolutionarily conserved low-affinity E2f binding site and ChIP assay performed in primary BM white blood cells shows that E2f1 and E2f3 bind to the Csf2rb promoter region (FIG. 5E), establishing Csf2rb as a non-canonical novel E2f target gene. Flow cytometry analysis confirms the increased expression of the common βsubunit receptor at the cell surface in TKO MPP2-4 subpopulations, but not in TKO LT-HSC-MPP1, as well as myeloid subpopulations, compared to CT (FIGS. 5F-G). Accordingly, exposure to Il3 increases the colony forming activity of TKO MPP3 but not TKO LT-HSCs, compared to CT counterparts (FIG. 5H). Merged analysis of scRNA-Seq data for MP and MPP4 populations from each genotype shows that Csf2rb expression is specifically increased in the most primitive cluster in TKO versus CT cells (FIG. 5I), which correlates with the increased expression of Pcna in that cluster. In contrast, Csf2ra and Il3ra, which codes for the α-chain receptor for Gm-Csf and Il3, respectively, display little variation in that cluster (not shown).

To functionally determine the consequences of increased Csf2rb and enhanced βcytokine signaling in TKO MPP4, we first knocked-down Csf2rb in CT and TKO MPP4 prior to their plating in methylcellulose. Silencing Csf2rb had no effect on CT MPP4 colony forming activity but reversed the increase in colony forming activity caused by Rb family inactivation (FIG. 5J). Consistent with an enhanced βcytokine activity in TKO MPP4 cells, CSL311® (a small molecule inhibitor of the βchain receptor⁴⁸) only inhibits TKO MPP4 colony forming activity at higher dose compared to CT (FIG. 5K). Collectively, these data show that E2f activates the expression of Csf2rb to enhance βcytokine signaling activity in TKO MPP4.

### Example 6 Irf8 Is a Critical Effector of Enhanced Pcytokine Signaling in TKO MPP4

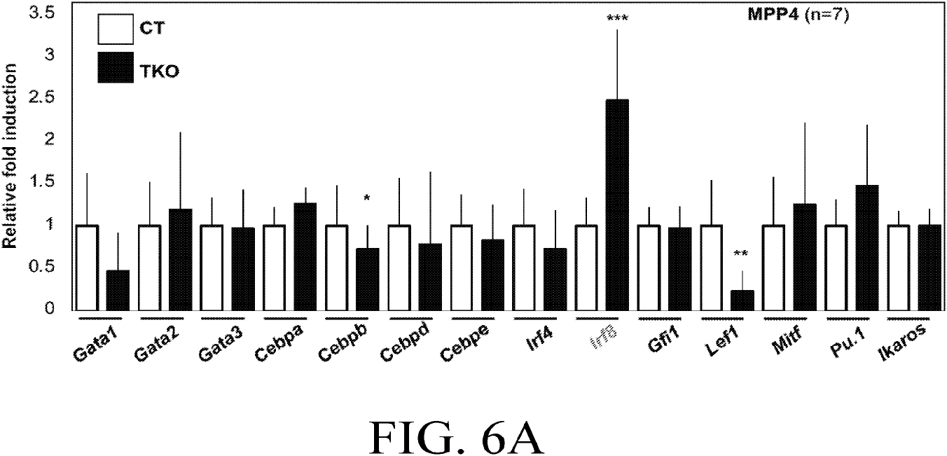

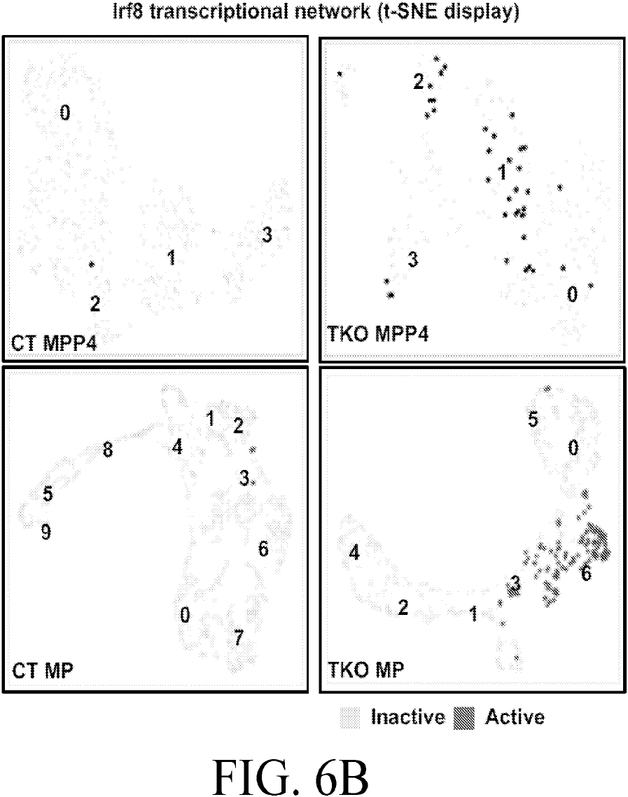

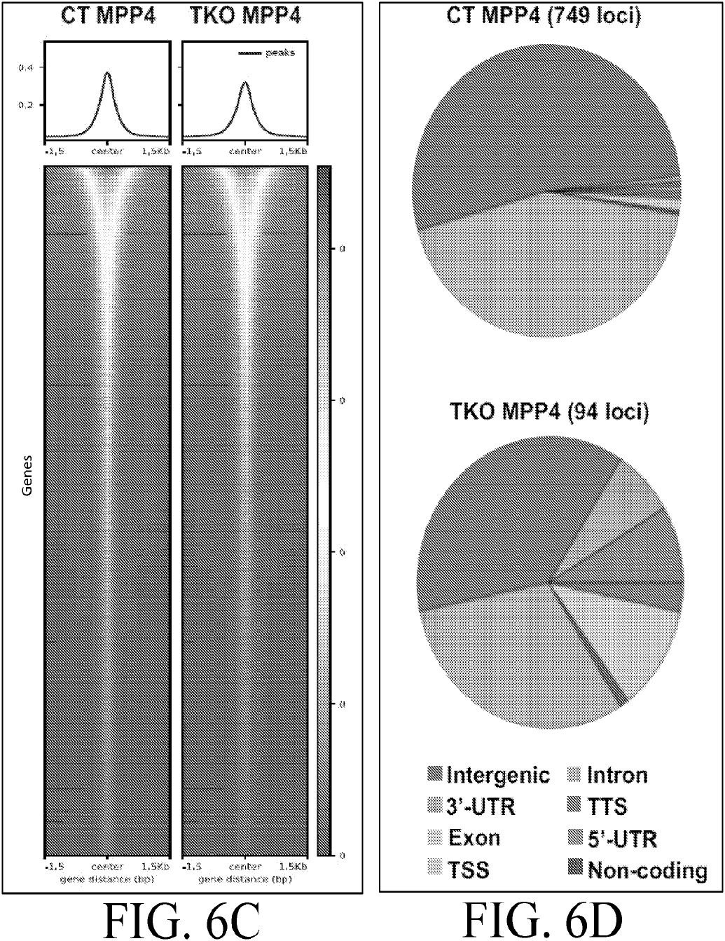

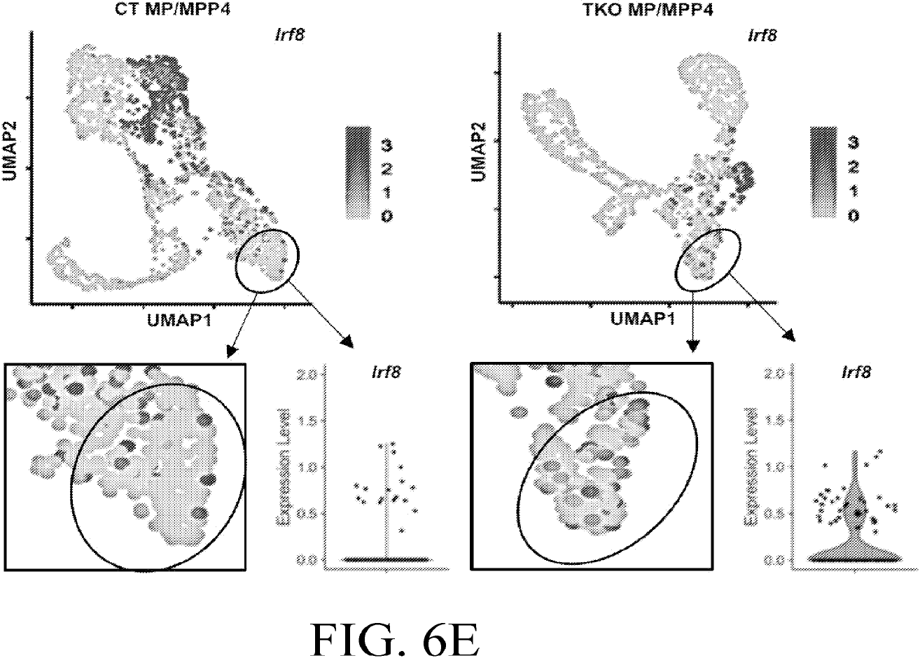

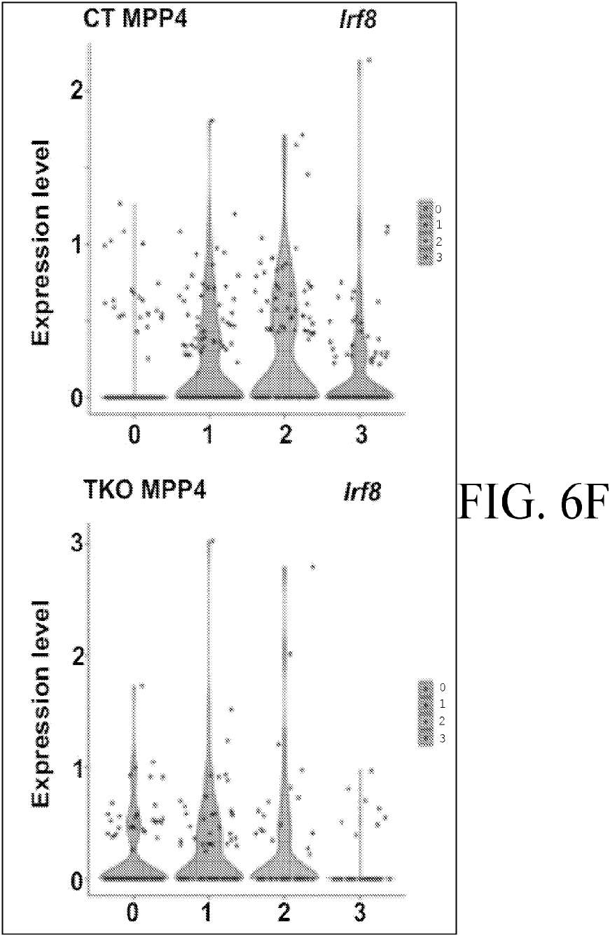

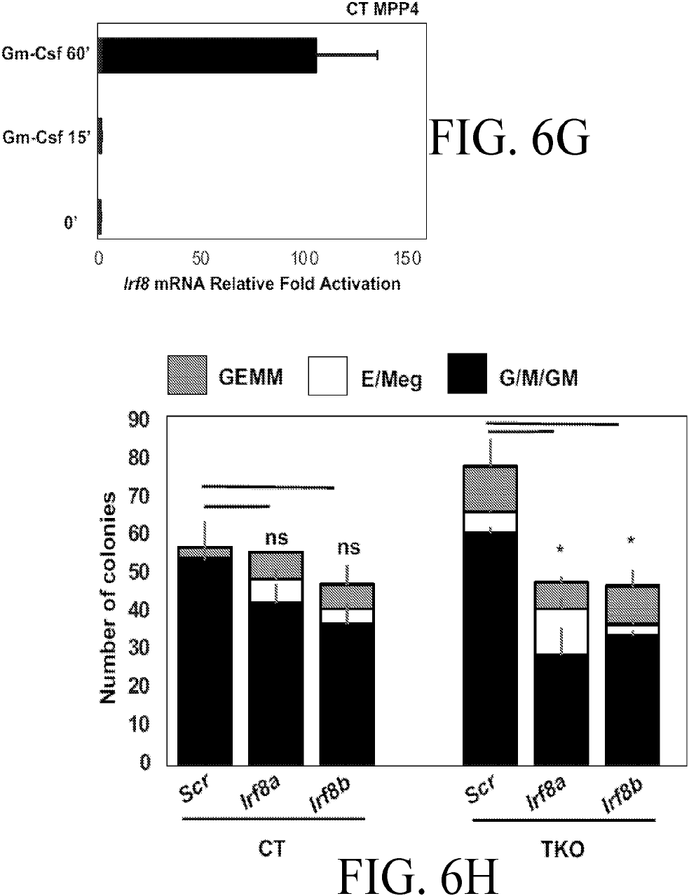

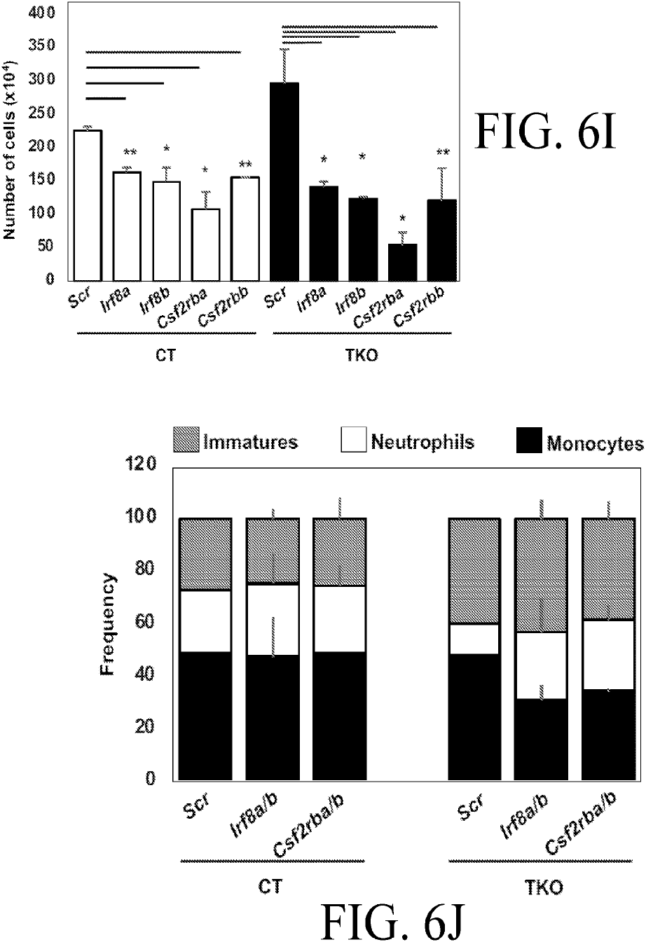

To identify the master effector(s) downstream of enhanced βcytokine signaling in TKO MPP4, we focused our attention on the transcription factors that govern hematopoiesis. A mini-screen RT-qPCR analysis identifies the increased expression of Irf8 in TKO MPP4 versus CT MPP4 (FIG. 6A) but not in TKO MPP3 versus CT MPP3, suggesting that Rb family inactivation triggers different transcriptional consequences in distinct MPP subpopulations. Irf8 is a critical promoter of monocyte and eosinophil lineages⁴⁹⁻⁵², which is consistent with the increased frequency of monocytes and eosinophils upon Rb family inactivation⁶. SCENIC⁴⁰ analysis shows minimal activity for the Irƒ8 transcriptional network in CT MPP4. In contrast, Irƒ8 transcriptional network is active in clusters 0, 1 and 2 from TKO MPP4, suggesting a role for Irf8 throughout the different steps of TKO MPP4 hierarchy (FIG. 6B). Similarly, Irf8 transcriptional network activity is also increased in TKO MPs (in particular in primitive clusters 3 and 6) compared to CT MPs (FIG. 6B). ATAC-Seq analysis shows mostly similar chromatin conformation between CT and TKO MPP4 populations (FIG. 6C). Of note, CT MPP4 have more uniquely-accessible chromatin regions compared to TKO MPP4 (749 vs 94 regions, respectively), suggesting that the lineage skewing of TKO MPP4 is accompanied by the discrete closing of a limited number of chromatin regions. Among the 94 regions that are uniquely accessible in TKO MPP4, coding regions were overrepresented compared to CT MPP4 (FIG. 6D). These data suggest that Irf8 activity triggers transcriptional variations in the absence of significant chromatin landscape remodeling in TKO MPP4. scRNA-Seq analysis shows that Irƒ8 expression is specifically increased in the most primitive cluster of TKO MPP4 compared to their controls, which correlates with the increased expression of Csf2rb (and Pcna) in this cluster (FIGS. 6E-6F). Irƒ8 regulatory regions contains functional binding sites for Stat and Nf-κB⁵³, two pathways downstream of βcytokine signaling. Accordingly, stimulation of MPP4 cells with Gm-Csf is sufficient to activate Irf8 (FIG. 6G), validating Irf8 as a βcytokine target gene in MPP4 cells. Irf8 silencing had no effect on the colony forming activity of CT MPP4 but, similar to Csf2rb silencing, reversed the increase in colony forming activity caused by Rb family inactivation (FIG. 6H), indicating a functional role for Irƒ8 in the altered contribution of TKO MPP4 to hematopoiesis. Silencing Irf8 or Csf2rb in TKO MPP4 comparably reversed the increase in cell growth (FIG. 6I) and the decreased neutrophil frequency (FIG. 6J) caused by Rb family inactivation. Collectively, these data identify Irƒ8 as a critical effector of βcytokine signaling in TKO MPP4.

### Example 7 Pcytokine Inhibition Represses Inflammatory Disease Development

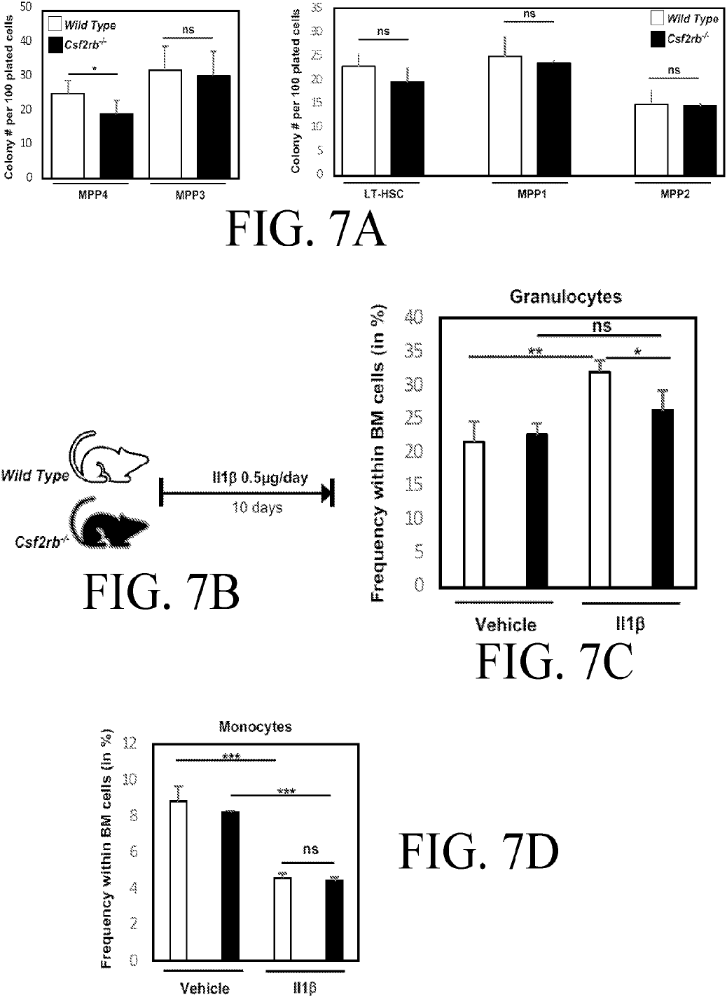

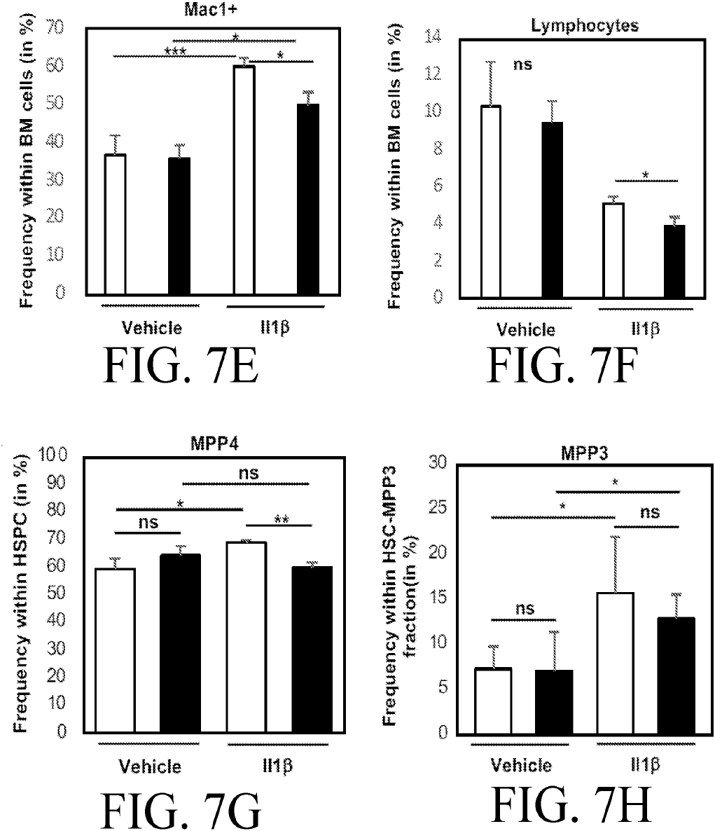

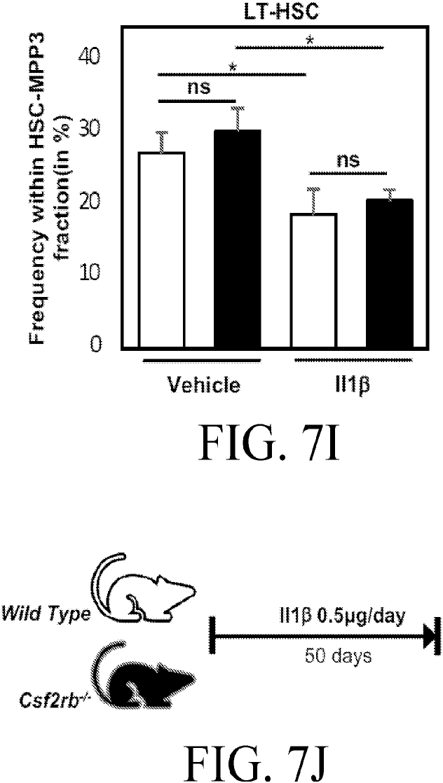

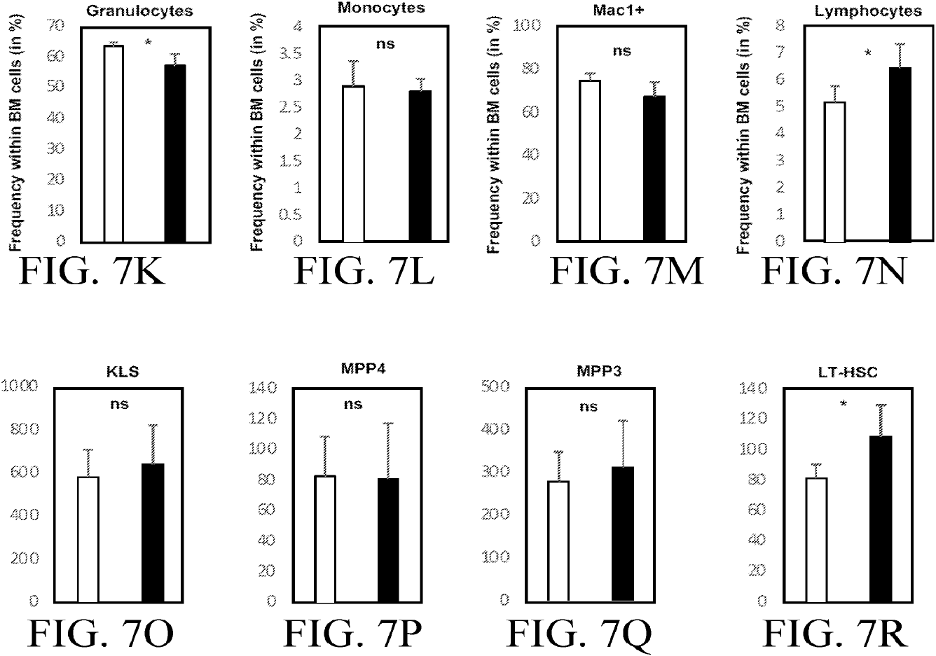

To determine the consequences of enhanced βcytokine activity for HSPC response to stress, HSPC subpopulations from wild-type and Csf2rb^{-/-} mice were first plated in methylcellulose to recapitulate a proliferative stress. Although βcytokine signaling is enhanced in both TKO MPP3 and MPP4 cells (FIG. 5), Csf2rb deficiency impairs the colony forming activity of MPP4 cells without altering the colony forming activity of MPP3 cells, indicating that MPP4 cells rely on βcytokine signaling for their response to stress (FIG. 7A). Furthermore, Csf2rb deficiency did not alter the colony forming activity of LT-HSC as well as MPP1-2 cells.

As a hallmark inflammatory cytokine, Il1β expression is increased in a large panel of inflammatory diseases ⁵⁴. To recapitulate a representative acute cytokine stress, wild type and Csf2rb^{-/-} mice were injected daily with recombinant Il1β for 10 days⁵⁵ (FIG. 7B). Granulocyte expansion in the bone marrow, as observed in wild-type mice, was inhibited by Csf2rb deficiency (FIGS. 7C-7E). In addition, B cell frequency decreases to a larger extent in Csf2rb^{-/-} mice compared to wild-type mice (FIG. 7F). Within the HSPC compartment, MPP4 frequency increases in Il1β-treated wild type mice, but not in Csf2rb^{-/-} mice (FIG. 7G). In contrast, exposure to Il1β increases the frequency of CT MPP3 and decreases the frequency of CT LT-HSCs, irrespective of Csf2rb expression status (FIGS. 7H-7I). Importantly, Csf2rb deficiency did not impact the frequency of mature and immature hematopoietic subpopulations in untreated mice, indicating that βcytokine signaling is dispensable in homeostatic conditions. To determine the consequences of bcytokine signaling inactivation in the context of chronic exposure to cytokine stress, wild type and Csf2rb^{-/-} mice were injected daily with recombinant Il1β for 50 days (FIG. 7J). Csf2rb deficiency led to decreased granulocyte frequency as well as increased lymphocyte frequency and LT-HSC abundance, respectively (FIGS. 7K-7R). Collectively, these data show that impaired βcytokine signaling preserves HSPC functions and limits granulocyte expansion in the bone marrow in the context of cytokine stress.

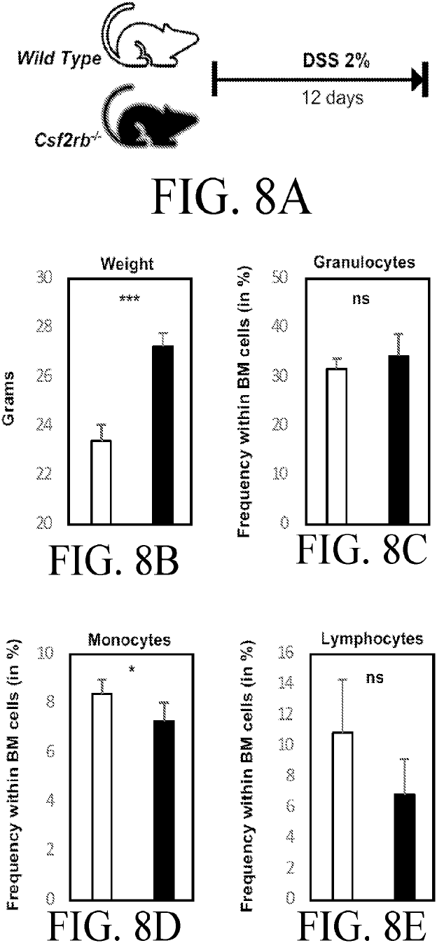

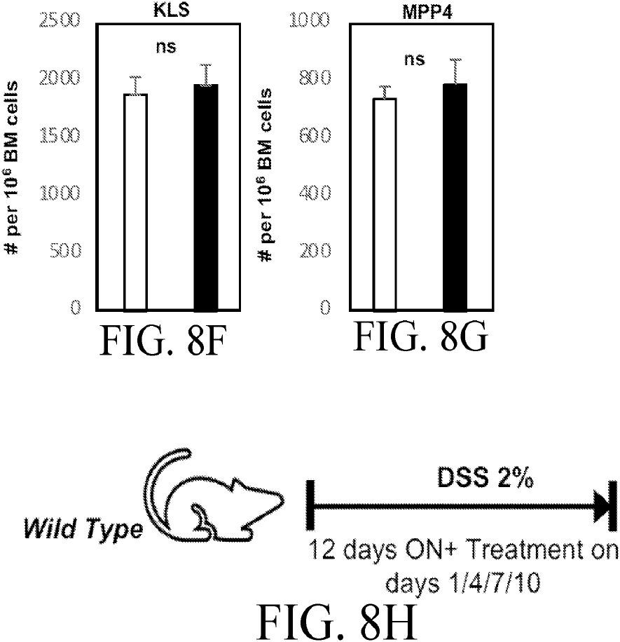

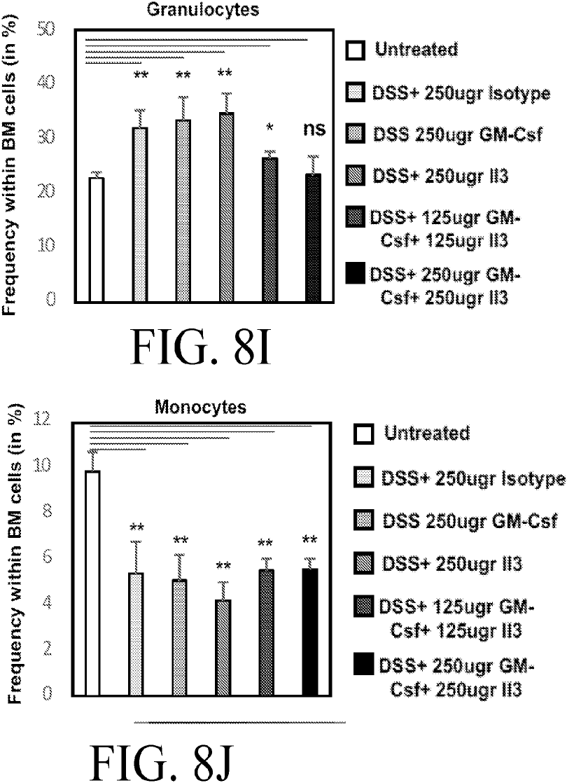

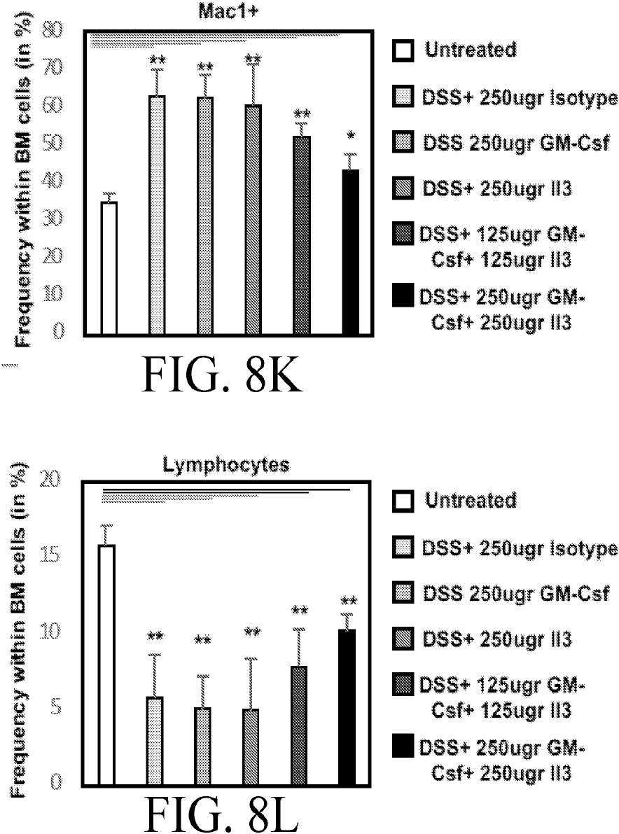

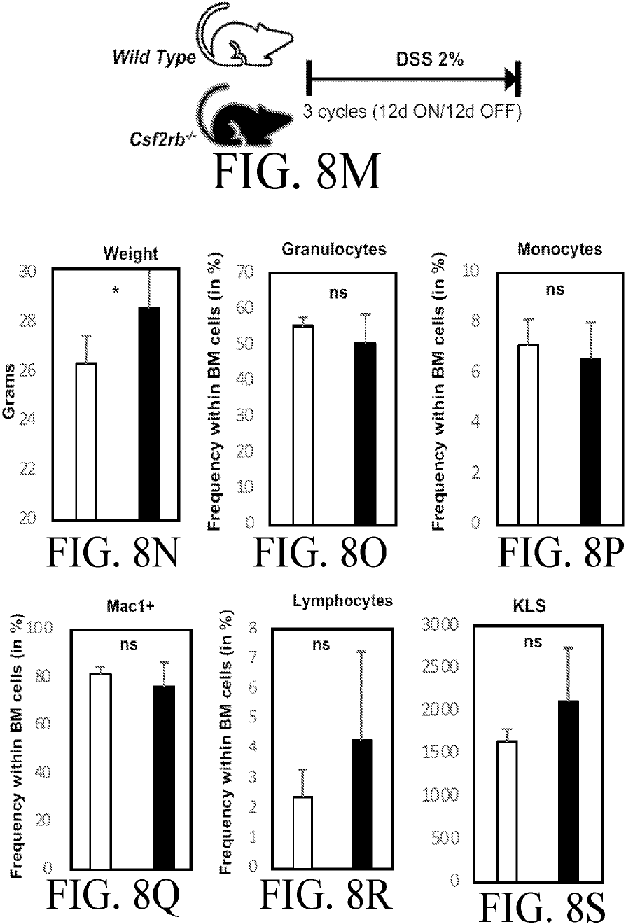

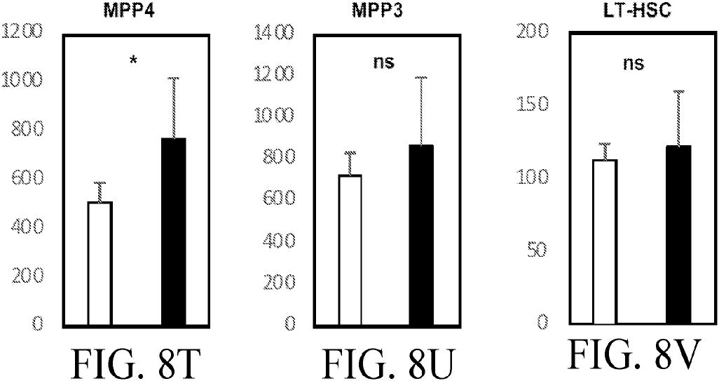

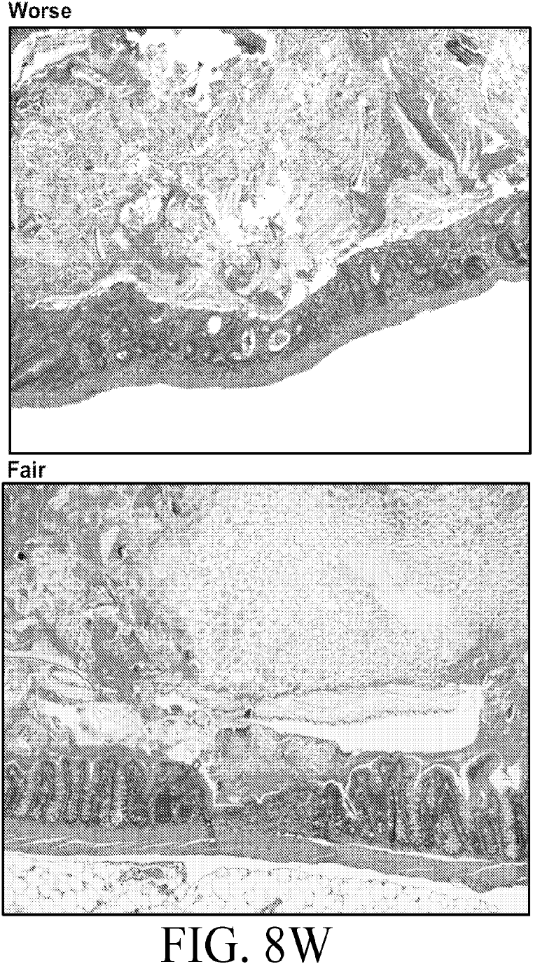

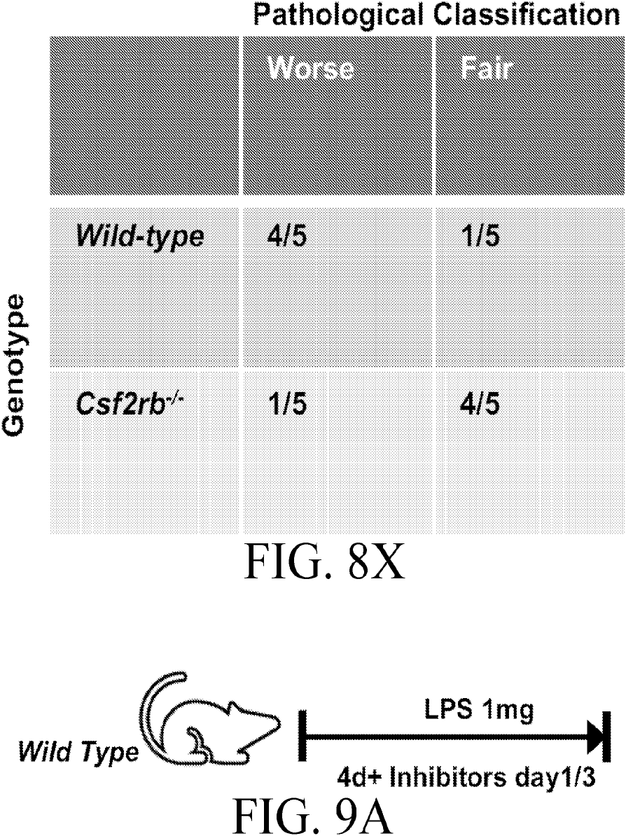

To determine the consequences of inhibiting βcytokine activity for inflammatory disease development, we first inhibited βcytokine signaling in a well-established preclinical model of inflammatory bowel disease (IBD). Cycles of addition/withdrawal of DSS (Dextran Sulfate Sodium) to drinking water recapitulates the clinical features of IBD. Acute exposure of wild type and Csf2rb^{-/-} mice to a 2% DSS regimen for 12 days (FIG. 8A) lead to a significant weight loss (a clinical feature of IBD diseases) in wild-type mice compared to Csf2rb deficient mice (FIG. 8B). Within hematopoietic subpopulations, Csf2rb deficiency leads to decreased monocyte frequency (FIGS. 8C-8G). In addition, wild-type mice exposed to 2% DSS regimen for 12 days were treated with different combinations of blocking antibodies for Gm-Csf and Il3 (FIG. 8H). Although single inhibition of either Gm-Csf or Il3 signaling did not affect myeloid expansion, combined Gm-Csf and Il3 inhibition decreased myeloid cell frequency and increased lymphopoiesis in a dose dependent manner (FIGS. 8I-8L). Collectively, these data indicate that genetic and pharmacological inhibition of βcytokine signaling activity represses stress myelopoiesis and improves clinical markers of IBD upon acute exposure to DSS. Exposure to a chronic IBD development regimen (FIG. 8M) also lead to a significant weight loss (FIG. 8N) and decreased MPP4 frequency (FIGS. 8O-8V) in wild-type mice compared to Csf2rb^{-/-} mice. Finally, histological analysis of different segments from the GI tract identified more foci of regeneration (indicative of tissue damage) in wild-type mice compared to Csf2rb^{-/-} mice (FIGS. 8W-8X). Collectively, these results indicate that βcytokine signaling inhibition represses IBD development.

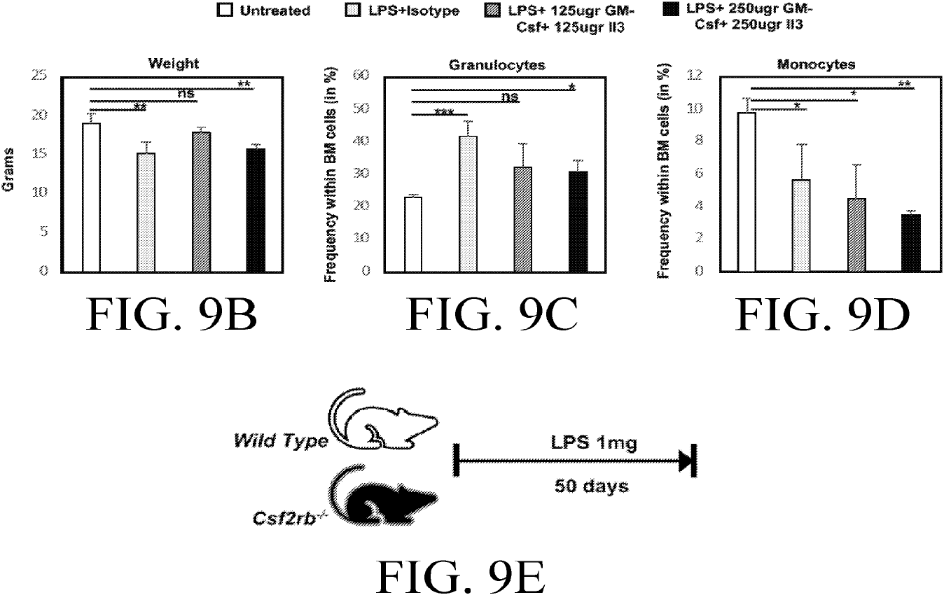

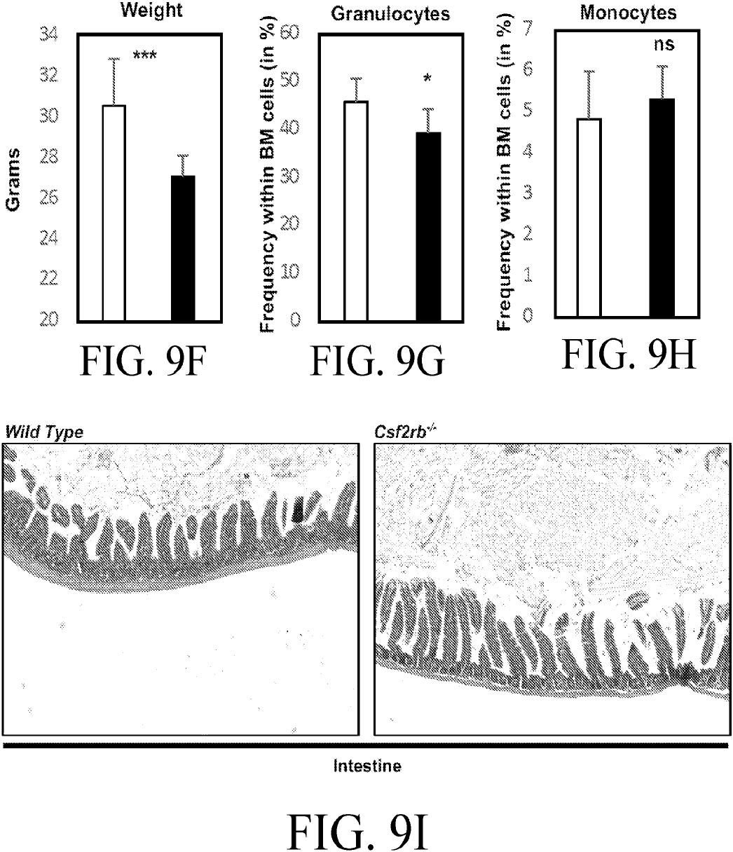

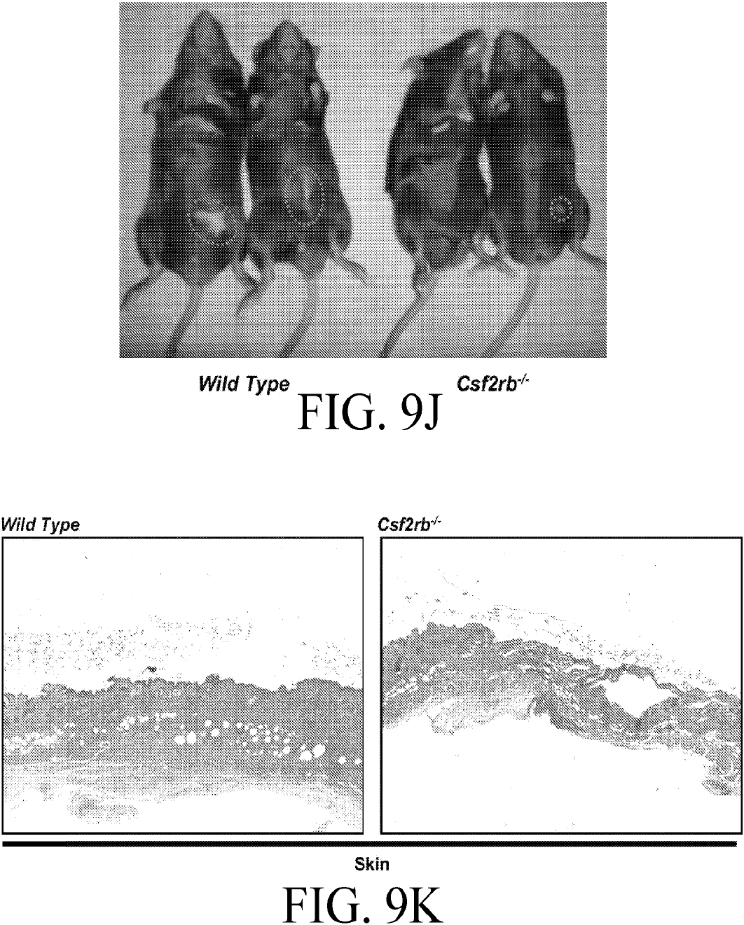

To determine the consequences of βcytokine signaling inhibition in the context of a systemic infection, mice were exposed to different regimens of LPS (a macromolecule present in the outer membrane of Gram- bacteria) injection. In the context of acute exposure to LPS treatment (FIG. 9A), combined pharmacological inhibition of βcytokine activity rescues weight loss and prevents granulocyte expansion in the bone marrow. In the context of chronic exposure to LPS treatment (FIG. 9E), Csf2rb deficiency prevents abnormal weight gain (a clinical feature of chronic inflammation) and decreases granulocyte expansion in the bone marrow (FIG. 9F-9H). In addition, Csf2rb deficiency restores normal villae structure in the intestine (FIG. 9I) and limits the development of scar tissue at the site of injection (FIGS. 9J-9K). Collectively, these results indicate that bcytokine signaling inhibition represses the consequences of LPS-induced systemic inflammation.

### Example 8 Treatment of Stress Hematopoiesis in Human Subjects

The results presented in the previous examples indicate that hematopoietic stress responses can be modulated by inhibiting βcytokine activity, providing a new therapeutic target to treat patients suffering for the consequences of stress hematopoiesis. Potential causes of stress hematopoiesis include without limitation, exposure to infection, auto-immune disease, cystic fibrosis, inflammatory diseases, aging, hypercholesterolemia, myocardial infarction, ischemia, and cancer.

A preferred embodiment of the invention comprises clinical application of the information described herein to a patient. This can occur after a patient arrives in the clinic and presents with symptoms of stress hematopoiesis.

As mentioned previously, treatment can entail the administration of βcytokine inhibitors such as Csl311, Csl362 and Mavrilimumab, alone or in combination, to inhibit the production of myeloid cells and unwanted aberrant immune activity. In addition to the βcytokine inhibitors, patients can also be treated with NSAIDS for general inflammation.

The skilled clinician would be aware of the proper dosing and schedule for treatment as clinical trials are currently being conducted to assess the efficacy of these agents in other disorders.

Csl311 https://clinicaltrials.gov/ct2/show/study/NCT04082754 and

Mavrilimumab https://www.ncbi.nlm.nih.gov/pmc/articles/PMC5947536/

References
1 Burkhart, D. L. & Sage, J. Cellular mechanisms of tumour suppression by the retinoblastoma gene. Nat Rev Cancer 8, 671-682, doi:nrc2399 [pii] 10.1038/nrc2399 (2008).
2 Chen, H. Z., Tsai, S. Y. & Leone, G. Emerging roles of E2Fs in cancer: an exit from cell cycle control. Nat Rev Cancer 9, 785-797, doi:10.1038/nrc2696 (2009).
3 Viatour, P. & Sage, J. Newly identified aspects of tumor suppression by RB. Dis Model Mech 4, 581-585, doi:10.1242/dmm.008060 (2011).
4 Julian, L. M. & Blais, A. Transcriptional control of stem cell fate by E2Fs and pocket proteins. Front Genet 6, 161, doi:10.3389/fgene.2015.00161 (2015).
5 Kim, E. et al. Rb family proteins enforce the homeostasis of quiescent hematopoietic stem cells by repressing Socs3 expression. J Exp Med 214, 1901-1912, doi:10.1084/jem.20160719 (2017).
6 Viatour, P. et al. Hematopoietic stem cell quiescence is maintained by compound contributions of the retinoblastoma gene family. Cell Stem Cell 3, 416-428, doi:10.1016/j.stem.2008.07.009 (2008).
7 Tarangelo, A. et al. Recruitment of Pontin/Reptin by E2f1 amplifies E2f transcriptional response during cancer progression. Nat Commun 6, 10028, doi:10.1038/ncomms10028 (2015).
8 Seita, J. & Weissman, I. L. Hematopoietic stem cell: self-renewal versus differentiation. Wiley Interdiscip Rev Syst Biol Med 2, 640-653, doi:10.1002/wsbm.86 (2010).
9 Adolfsson, J. et al. Identification of Flt3+ lympho-myeloid stem cells lacking erythro-megakaryocytic potential a revised road map for adult blood lineage commitment. Cell 121, 295-306, doi:10.1016/cell.2005.02.013 (2005).
10 Cabezas-Wallscheid, N. et al. Identification of regulatory networks in HSCs and their immediate progeny via integrated proteome, transcriptome, and DNA methylome analysis. Cell Stem Cell 15, 507-522, doi:10.1016/j.stem.2014.07.005 (2014).
11 Forsberg, E. C., Serwold, T., Kogan, S., Weissman, I. L. & Passegue, E. New evidence supporting megakaryocyte-erythrocyte potential of flk2/flt3+ multipotent hematopoietic progenitors. Cell 126, 415-426, doi:10.1016/j.cell.2006.06.037 (2006).
12 Wilson, A. et al. Hematopoietic stem cells reversibly switch from dormancy to self-renewal during homeostasis and repair. Cell 135, 1118-1129, doi:10.1016/j.cell.2008.10.048 (2008).
13 Monticelli, S. & Natoli, G. Transcriptional determination and functional specificity of myeloid cells: making sense of diversity. Nat Rev Immunol 17, 595-607, doi:10.1038/nri.2017.51 (2017).
14 Balla, K. M. et al. Eosinophils in the zebrafish: prospective isolation, characterization, and eosinophilia induction by helminth determinants. Blood 116, 3944-3954, doi:10.1182/blood-2010-03-267419 (2010).
15 Ohmori, S. et al. GATA2 is critical for the maintenance of cellular identity in differentiated mast cells derived from mouse bone marrow. Blood 125, 3306-3315, doi:10.1182/blood-2014-11-612465 (2015).
16 Iwasaki, H. et al. The order of expression of transcription factors directs hierarchical specification of hematopoietic lineages. Genes Dev 20, 3010-3021, doi:10.110⅟gad.1493506 (2006).
17 Vainchenker, W. & Kralovics, R. Genetic basis and molecular pathophysiology of classical myeloproliferative neoplasms. Blood 129, 667-679, doi:10.1182/blood-2016-10-695940 (2017).
18 Ohlsson, E., Schuster, M. B., Hasemann, M. & Porse, B. T. The multifaceted functions of C/EBPalpha in normal and malignant haematopoiesis. Leukemia 30, 767-775, doi:10.1038/leu.2015.324 (2016).
19 Rosenbauer, F. & Tenen, D. G. Transcription factors in myeloid development: balancing differentiation with transformation. Nat Rev Immunol 7, 105-117, doi:10.1038/nri2024 (2007).
20 Courties, G. et al. Ischemic stroke activates hematopoietic bone marrow stem cells. Circ Res 116, 407-417, doi:10.116⅟CIRCRESAHA.116.305207 (2015).
21 Heidt, T. et al. Chronic variable stress activates hematopoietic stem cells. NatMed 20, 754-758, doi:10.1038/nm.3589 (2014).
22 Murphy, A. J. et al. ApoE regulates hematopoietic stem cell proliferation, monocytosis, and monocyte accumulation in atherosclerotic lesions in mice. J Clin Invest 121,4138-4149, doi:10.1172/JCI57559 (2011).
23 Wang, M. et al. Interleukin-3/granulocyte macrophage colony-stimulating factor receptor promotes stem cell expansion, monocytosis, and atheroma macrophage burdenin mice with hematopoietic ApoE deficiency. Arterioscler Thromb Vasc Biol 34, 976-984, doi:10.116⅟ATVBAHA.113.303097 (2014).
24 Yvan-Charvet, L. et al. ATP-binding cassette transporters and HDL suppress hematopoietic stem cell proliferation. Science 328, 1689-1693, doi:10.1126/science.1189731 (2010).
25 Snoeck, H. W. Aging of the hematopoietic system. Curr Opin Hematol 20, 355-361, doi:10.1097/MOH.0b013e3283623c77 (2013).
26 Pietras, E. M. Inflammation: a key regulator of hematopoietic stem cell fate in health and disease. Blood 130, 1693-1698, doi:10.1182/blood-2017-06-780882 (2017).
27 Mangalmurti, N. & Hunter, C. A. Cytokine Storms: Understanding COVID-19. Immunity 53, 19-25, doi:10.1016/j.immuni.2020.06.017 (2020).
28 Schultze, J. L., Mass, E. & Schlitzer, A. Emerging Principles in Myelopoiesis at Homeostasis and during Infection and Inflammation. Immunity 50, 288-301, doi:10.1016/j.immuni.2019.01.019 (2019).
29 Zhao, J. L. & Baltimore, D. Regulation of stress-induced hematopoiesis. Curr Opin Hematol 22, 286-292, doi:10.1097/MOH.0000000000000149 (2015).
30 Cheng, T. et al. Hematopoietic stem cell quiescence maintained by p21cip⅟waf1. Science 287, 1804-1808 (2000).
31 Matsumoto, A. et al. p57 is required for quiescence and maintenance of adult hematopoietic stem cells. Cell Stem Cell 9, 262-271, doi:10.1016/j.stem.2011.06.014 (2011).
32 Zou, P. et al. p57(Kip2) and p27(Kip1) cooperate to maintain hematopoietic stem cell quiescence through interactions with Hsc70. Cell Stem Cell 9, 247-261, doi:10.1016/j.stem.2011.07.003 (2011).
33 Yilmaz, O. H. et al. Pten dependence distinguishes haematopoietic stem cells from leukaemia-initiating cells. Nature 441, 475-482, doi:10.1038/nature04703 (2006).
34 Baumgartner, C. et al. An ERK-Dependent Feedback Mechanism Prevents Hematopoietic Stem Cell Exhaustion. Cell Stem Cell 22, 879-892.e876, doi:10.1016/j.stem.2018.05.003 (2018).
35 Tothova, Z. et al. FoxOs are critical mediators of hematopoietic stem cell resistance to physiologic oxidative stress. Cell 128, 325-339, doi:10.1016/j.cell.2007.01.003 (2007).
36 Mende, N. et al. CCND1-CDK4-mediated cell cycle progression provides a competitive advantage for human hematopoietic stem cells in vivo. J Exp Med 212, 1171-1183, doi:10.1084/jem.20150308 (2015).
37 Bernitz, J. M., Kim, H. S., MacArthur, B., Sieburg, H. & Moore, K. Hematopoietic Stem Cells Count and Remember Self-Renewal Divisions. Cell 167, 1296-1309.e1210, doi:10.1016/j.cell.2016.10.022 (2016).
38 Lauridsen, F. K. B. et al. Differences in Cell Cycle Status Underlie Transcriptional Heterogeneity in the HSC Compartment. Cell Rep 24, 766-780, doi:10.1016/j.celrep.2018.06.057 (2018).
39 Orford, K. W. & Scadden, D. T. Deconstructing stem cell self-renewal: genetic insights into cell-cycle regulation. Nat Rev Genet 9, 115-128, doi:10.1038/nrg2269 (2008).
40 Aibar, S. et al. SCENIC: single-cell regulatory network inference and clustering. Nat Methods 14, 1083-1086, doi:10.1038/nmeth.4463 (2017).
41 Busch, K. et al. Fundamental properties of unperturbed haematopoiesis from stem cells in vivo. Nature 518, 542-546, doi:10.1038/nature14242 (2015).
42 Paul, F. et al. Transcriptional Heterogeneity and Lineage Commitment in Myeloid Progenitors. Cell 163, 1663-1677, doi:10.1016/j.cell.2015.11.013 (2015).
43 Yanez, A. et al. Granulocyte-Monocyte Progenitors and Monocyte-Dendritic Cell Progenitors Independently Produce Functionally Distinct Monocytes. Immunity 47, 890-902 e894, doi:10.1016/j.immuni.2017.10.021 (2017).
44 Giladi, A. et al. Single-cell characterization of haematopoietic progenitors and their trajectories in homeostasis and perturbed haematopoiesis. Nat Cell Biol 20, 836-846, doi:10.1038/s41556-018-0121-4 (2018).
45 Broughton, S. E. et al. The betac receptor family - Structural insights and their functional implications. Cytokine 74, 247-258, doi:10.1016/j.cyto.2015.02.005 (2015).
46 Hercus, T. R. et al. Signalling by the betac family of cytokines. Cytokine Growth Factor Rev 24, 189-201, doi:10.1016/j.cytogfr.2013.03.002 (2013).
47 Hercus, T. R. et al. Role of the beta Common (betac) Family of Cytokines in Health and Disease. Cold Spring Harb Perspect Biol, doi:10.110⅟cshperspect.a028514 (2017).
48 Panousis, C. et al. CSL311, a novel, potent, therapeutic monoclonal antibody for the treatment of diseases mediated by the common beta chain of the IL-3, GM-CSF and IL-5 receptors. MAbs 8, 436-453, doi:10.1080/19420862.2015.1119352 (2016).
49 Sasaki, H. et al. Transcription factor IRF8 plays a critical role in the development of murine basophils and mast cells. Blood 125, 358-369, doi:10.1182/blood-2014-02-557983 (2015).
50 Tamura, T., Kurotaki, D. & Koizumi, S. Regulation of myelopoiesis by the transcription factor IRF8. Int J Hematol 101, 342-351, doi:10.1007/s12185-015-1761-9 (2015).
51 Yanez, A., Ng, M. Y., Hassanzadeh-Kiabi, N. & Goodridge, H. S. IRF8 acts in lineage-committed rather than oligopotent progenitors to control neutrophil vs monocyte production. Blood 125, 1452-1459, doi:10.1182/blood-2014-09-600833 (2015).
52 Yanez, A. & Goodridge, H. S. Interferon regulatory factor 8 and the regulation of neutrophil, monocyte, and dendritic cell production. Curr Opin Hematol 23, 11-17, doi:10.1097/MOH.0000000000000196 (2016).
53 Kanno, Y. et al. The genomic structure of the murine ICSBP gene reveals the presence of the gamma interferon-responsive element, to which an ISGF3 alpha subunit (or similar) molecule binds. Mol Cell Biol 13, 3951-3963 (1993).
54 Garlanda, C., Dinarello, C. A. & Mantovani, A. The interleukin-1 family: back to the future. Immunity 39, 1003-1018, doi:10.1016/j.immuni.2013.11.010 (2013).
55 Pietras, E. M. et al. Chronic interleukin-1 exposure drives haematopoietic stem cells towards precocious myeloid differentiation at the expense of self-renewal. Nat Cell Biol 18, 607-618, doi:10.1038/ncb3346 (2016).
56 Xu, X. et al. A comprehensive ChIP-chip analysis of E2F1, E2F4, and E2F6 in normal and tumor cells reveals interchangeable roles of E2F family members. Genome Res 17, 1550-1561, doi:10.110⅟gr.6783507 (2007).
57 Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218, doi:10.1038/nmeth.2688 (2013).
58 Zhang, Y. et al. Model-based analysis of ChIP-Seq (MACS). Genome Biol 9, R137, doi:10.1186/gb-2008-9-9-r137 (2008).
59 Heinz, S. et al. Simple combinations of lineage-determining transcription factors prime cis-regulatory elements required for macrophage and B cell identities. Mol Cell 38, 576-589, doi:10.1016/j.molcel.2010.05.004 (2010).
60 Ramírez, F. et al. deepTools2: a next generation web server for deep-sequencing data analysis. Nucleic Acids Res 44, W160-165, doi:10.1093/nar/gkw257 (2016).
61 Stuart, T. et al. Comprehensive Integration of Single-Cell Data. Cell 177, 1888-1902.e1821, doi: 10.1016/j.cell.2019.05.031 (2019).
62 Qiu, X. et al. Reversed graph embedding resolves complex single-cell trajectories. Nat Methods 14, 979-982, doi:10.1038/nmeth.4402 (2017).
63 Cao, J. et al. The single-cell transcriptional landscape of mammalian organogenesis. Nature 566, 496-502, doi:10.1038/s41586-019-0969-x (2019).
64 La Manno, G. et al. RNA velocity of single cells. Nature 560, 494-498, doi: 10.1038/s41586-018-0414-6 (2018).
65 Pietras, E. M. et al. Functionally Distinct Subsets of Lineage-Biased Multipotent Progenitors Control Blood Production in Normal and Regenerative Conditions. Cell Stem Cell 17, 35-46, doi:10.1016/j.stem.2015.05.003 (2015).

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for treating stress hematopoiesis in a subject in need thereof comprising contacting multipotent progenitor (MPP) cells and their progeny with at least one agent that represses formation and activity of myeloid cells generated from differentiation of said MPP cells, thereby alleviating the symptoms of stress hematopoiesis in said subject.

2. The method of claim 1, wherein the MPP cells are one or both of MPP3 and MPP4 cells.

3. The method of claim 1, wherein said agent inhibits βcytokine activity.

4. The method of claim 3, wherein said agent blocks or inhibits the common β chain of the β cytokine receptor.

5. The method of claim 3, wherein said agent blocks or inhibits the cytokine specific α chain of the β cytokine receptor.

6. The method of claim 3, wherein said agent is an inhibitory nucleic acid which targets the Csf2rb gene.

7. The method of claim 3, wherein said agent is an inhibitory nucleic acid which targets the cytokine-specific α chain gene of the β cytokine receptor.

8. The method of claim 6, wherein said inhibitory nucleic acid is selected from an siRNA, an shRNA, and an antisense oligonucleotide.

9. The method of claim 3, wherein βcytokine activity is inhibited via administration of at least one antibody which blocks IL3 binding to the α chain, an antibody that blocks Gm-CsF binding to the α chain or both.

10. The method of claim 3, wherein said agent is at least one small molecule βcytokine inhibitor.

11. The method of claim 1 wherein said subject has a condition selected from systemic infection, chronic inflammation, lupus, Inflammatory Bowel Disease, aging, hypercholesterolemia, myocardial infarction, ischemia, cancer, cystic fibrosis and arthritis, wherein said treatment modulates hematopoiesis providing therapeutic benefit to the subject.

12. The method of claim 10 wherein said βcytokine inhibitor is selected from one or more of Csl311, Csl362, and Mavrilimumab.

13. The method of claim 1, further comprising administration of at least one NSAIDS.

14. The method of claim 10 wherein Csl362 and Mavrilimumab are administered.

15. The method of claim 10 wherein Csl311 is administered.

16. The method of claim 7, wherein said inhibitory nucleic acid is selected from an siRNA, an shRNA, and an antisense oligonucleotide.

17. The method of claim 12 wherein said subject has a condition selected from systemic infection, chronic inflammation, lupus, Inflammatory Bowel Disease, aging, hypercholesterolemia, myocardial infarction, ischemia, cancer, cystic fibrosis and arthritis, wherein said treatment modulates hematopoiesis providing therapeutic benefit to the subject.
